(19) United States
(12) Patent Application Publication
(10) Pub. No.: US 2024/0366454 A1
(43) Pub. Date: Nov. 7, 2024

(54) VACUUM PAD AND RELATED SYSTEM

(71) Applicant: ORLANDO HEALTH, INC., ORLANDO, FL (US)

(72) Inventors: Teresa Lance, ORLANDO, FL (US); Neeraj Desai, Windmere, FL (US); Neil Whitehouse, North Kingstown, RI (US); Michael J. Schmidt, ORLANDO, FL (US); Riham Mikhail Hanna, Winter Park, FL (US); Anthony Mango, Orlando, FL (US); Evan Anthony Mulone, Marston's Mills, MA (US); Luis Ruben Marquez, Providence, RI (US); David Anthony Lefort, Providence, RI (US); Breno Cloutier Dobal, Orlando, FL (US); Emily Frier, Clermont, FL (US); Izayha Woods, Apopka, FL (US); Khanh Tran, Orlando, FL (US); Jung Woo Lee, Oviedo, FL (US); Sonia Alvarez, Boca Raton, FL (US)

(73) Assignee: ORLANDO HEALTH, INC., ORLANDO, FL (US)

(21) Appl. No.: 18/775,883

(22) Filed: Jul. 17, 2024

Related U.S. Application Data
(63) Continuation-in-part of application No. 17/819,846, filed on Aug. 15, 2022, now Pat. No. 12,042,438.
Application No. 17/819,846 is a continuation of application No. 17/499,346, filed on Oct. 12, 2021, now Pat. No. 11,413,206.

(51) Int. Cl. A61G 13/10; A61G 13/00; A61M 1/00

(52) U.S. Cl. CPC A61G 13/102; A61G 13/0009; A61M 1/73; A61G 2203/30

(57) ABSTRACT

A vacuum pad may include a first layer, a top layer that at least partially forms a top surface of the vacuum pad, a plurality of holes extending through the top layer into an interior space located between at least a portion of the first layer and at least a portion of the top layer, an outlet tube in fluid communication with the interior space, a raised barrier that surrounds the plurality of holes, a plurality of dividers located in the interior space that extend along the first layer from a first end of the respective divider located adjacent to the outlet tube, and at least one spacer located in the interior space that has a different shape than the plurality of dividers. The vacuum pad may include at least one channel and at least one collection area located in the interior space.

### RELATED APPLICATION(S)

This application is a continuation-in-part of U.S. patent application Ser. No. 17/819,846, filed on Aug. 15, 2022, entitled “VACUUM PAD AND RELATED SYSTEM,” which is a continuation of U.S. patent application Ser. No. 17/499,346, filed Oct. 12, 2021 (now U.S. Pat. No. 11,413,206), entitled “VACUUM PAD AND RELATED SYSTEM.” The applications and patents listed in this paragraph are hereby incorporated by reference in their entirety.

### TECHNICAL FIELD

The present disclosure relates to a device for collecting and monitoring fluid loss in a medical procedure, such as a mother's blood loss during a caesarean delivery. In particular, the present disclosure relates to a vacuum pad and related system.

### BACKGROUND

A human or animal patient may experience fluid loss in a variety of situations, particularly in surgical procedures. For instance, some women experience moderate to severe bleeding from the uterus during a Cesarean delivery (“C-section”). While a certain amount of bleeding is normal and safe, (e.g., an average C-section is associated with about four cups of blood loss), excess blood loss can be dangerous. Other fluid losses that may need to be addressed included losses of amniotic fluid, vaginal discharge, etc. To complicate matters, the patient is often covered by a surgical drape, which may limit the surgeon's ability to monitor fluid loss visually. To address these issues, the present disclosure relates to a device for collecting and monitoring fluid loss in a medical procedure.

### BRIEF DESCRIPTION OF THE DRAWINGS

The embodiments discussed herein may be better understood with reference to the following drawings and description. The components in the figures are not necessarily to scale. Moreover, in the figures, like-referenced numerals designate corresponding parts throughout the different views.

FIG. 1 is an illustration showing a vacuum system for collecting and monitoring a fluid in accordance with certain aspects of the present disclosure.

FIG. 2 is an illustration showing the vacuum system from FIG. 1 during a surgical procedure, where blood is being collected in accordance with certain aspects of the present disclosure.

FIG. 3 is an illustration showing an isolated view of the vacuum pad from the system of FIG. 1.

FIG. 4 is an illustration showing an exploded view of a portion of the vacuum pad from FIG. 3 having a multi-layer construction with spacers therein in accordance with certain aspects of the present disclosure.

FIG. 5 is an illustration showing a second embodiment of a vacuum pad having a tube extending through an interior space in accordance with certain aspects of the present disclosure.

FIG. 6 is an illustration showing an exploded view of a portion of the vacuum pad from FIG. 5 having a multi-layer construction with spacers therein in accordance with certain aspects of the present disclosure.

FIG. 7 is an illustration showing a third embodiment of a vacuum pad having a support structure extending from an outlet tube in accordance with certain aspects of the present disclosure.

FIG. 8 is an illustration showing a magnified view detailing the support structure from the embodiment of FIG. 7.

FIG. 9 is an illustration showing a fourth embodiment of a vacuum pad having a polygon active area and star opening pattern in accordance with certain aspects of the present disclosure.

FIG. 10 is an illustration showing a fifth embodiment of a vacuum pad having a square active area and grid opening pattern in accordance with certain aspects of the present disclosure.

FIG. 11 is an illustration showing a vacuum pad having an active area surrounded by a boundary area in accordance with certain aspects of the present disclosure.

FIG. 12 is an illustration showing a vacuum pad having an active area surrounded by a barrier in accordance with certain aspects of the present disclosure.

FIG. 13 is an illustration showing a vacuum pad having a sloped surface for directing a fluid in a certain direction in accordance with certain aspects of the present disclosure.

FIG. 14 is an illustration showing a vacuum pad being coupled to a funnel jig for directing fluid towards the vacuum pad in accordance with certain aspects of the present disclosure.

FIGS. 15A-15D are illustrations showing a vacuum pad having an outlet tube extending from different locations in accordance with certain aspects of the present disclosure.

FIGS. 16A-16B are illustrations showing a vacuum pad having multiple outlet tubes in accordance with certain aspects of the present disclosure.

FIGS. 17A-17D are illustrations showing a vacuum pad having multiple ports for receiving an outlet tube in accordance with certain aspects of the present disclosure.

FIGS. 18A-18C are illustrations showing an embodiment of a vacuum pad having at least one divider and an outlet pocket in accordance with certain aspects of the present disclosure.

FIGS. 19A-19B are illustrations showing an embodiment of a vacuum pad having one or more dividers and at least one channel in accordance with certain aspects of the present disclosure.

FIG. 20 is an illustration showing a perspective view of the embodiment depicted in FIGS. 19A-19B.

FIG. 21A is an illustration showing an exploded view of the embodiment depicted in FIGS. 19A-19B and 20.

FIG. 21B is an illustration showing a magnified view of the embodiment depicted in FIGS. 19A-19B, 20, and 21A.

FIG. 22 is an illustration showing an embodiment of a suspension system coupled to a canister, where the canister is configured to collect fluid from a vacuum pad, and the suspension system is configured to detect both total fluid loss and fluid loss rate.

### DETAILED DESCRIPTION

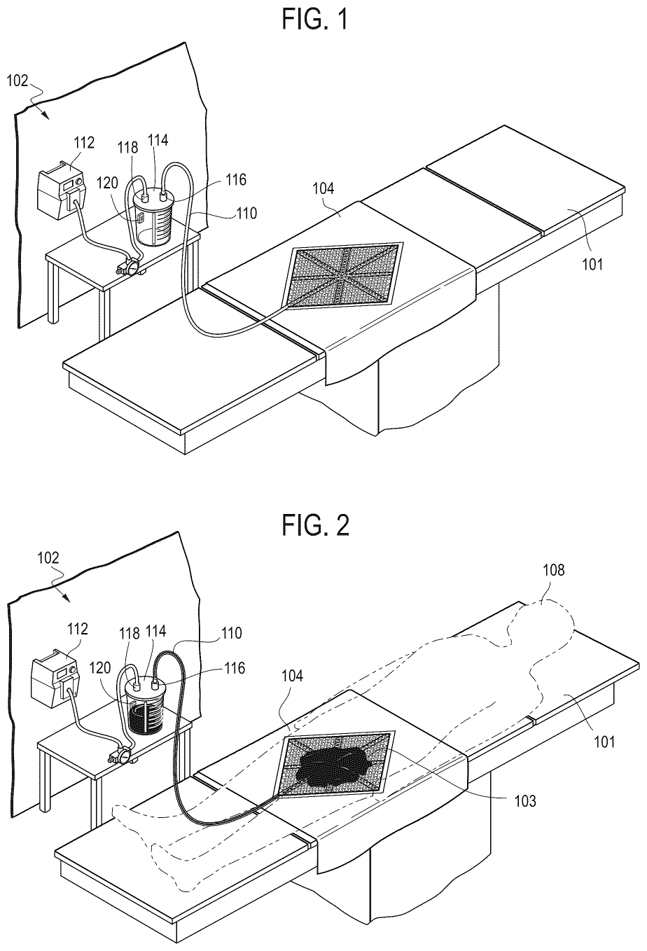

FIGS. 1-2 show an example of a vacuum fluid collection system 102. The system 102 generally includes a vacuum pad 104 being utilized for the collection of blood 103 or another fluid from a patient. The vacuum pad 104 and vacuum system 102 are described herein as being designed for use in a medical setting. However, it is recognized that these devices may additionally, or alternatively, be used in other contexts, including settings outside of medical applications.

In the depicted embodiment, the vacuum pad 104 and/or the remainder of the vacuum system 102 may be used for any suitable medical procedure, particularly where the collection and monitoring of a body fluid originating from a patient 108 are desired. For simplicity of description, this disclosure focuses on the collection and monitoring of blood 106 from a surgical table 101 during a C-section (e.g., FIG. 2). Since some women experience moderate to severe bleeding from the uterus during a C-section, it is desirable to collect the blood as it comes from the woman's body and to remove it from the top surface of the surgical table. Further, since excess blood loss can be dangerous, it is desirable to monitor the total amount of blood lost during the procedure as well as the real-time rate of loss. The devices and methods described herein accomplish both of these features.

The vacuum system 102 may generally include the vacuum pad 104, which is configured to be placed on the surgical table 101 and beneath a patient's body 108. As discussed in greater detail below, the vacuum pad 104 may be configured for the collection of blood (or another fluid) from the patient, and may direct that blood to a vacuum hose 110 to move the blood away from the surgical table 101. In other embodiments, the vacuum pad 104 may be a portion of the surgical table 101 (e.g., providing a portion of the support for the table's top surface).

The vacuum hose 110 may ultimately be in fluid communication with a vacuum source 112. Any suitable vacuum source 112 may be used. For example, the vacuum source 112 may be a common vacuum-supply system available in many hospital settings that comply with NFPA99 design standards, where a pump within a dedicated room of the building provides a vacuum to each (or many) of the hospital rooms via vacuum plumbing. In other applications, a portable, stand-alone vacuum source 112 may be used instead.

A canister 114 may be located between the vacuum pad 104 and the vacuum source 112, as shown. The canister 114 may be configured for collecting and storing the blood as it is pulled from the vacuum pad 104. For example, an inlet 116 to the canister 114, and also a vacuum outlet 118, may be located near a top portion of the canister 114. Advantageously, once blood enters from the top of the canister 114 via the inlet 116, it falls to the bottom of the canister 114 where it remains collected and stored for the duration of the procedure.

The total blood loss from the patient 108 may directly correlate with the volume and/or mass of blood collected within the canister 114. This may be monitored visually (e.g., with graduations on a transparent surface of the canister 114) and/or electrically. For example, a sensor 120 may be included with the canister 114 for detecting a characteristic of the blood or other fluid within the canister 114. In some embodiments, the sensor 120 may be a fluid level sensor that is fixed to the canister 114. In other embodiments, the sensor 120 may alternatively, or additionally, be a weight/mass scale placed beneath the canister 114 such that the mass of liquid within the canister 114 is monitored. Any other suitable sensor can be alternatively, or additionally, included.

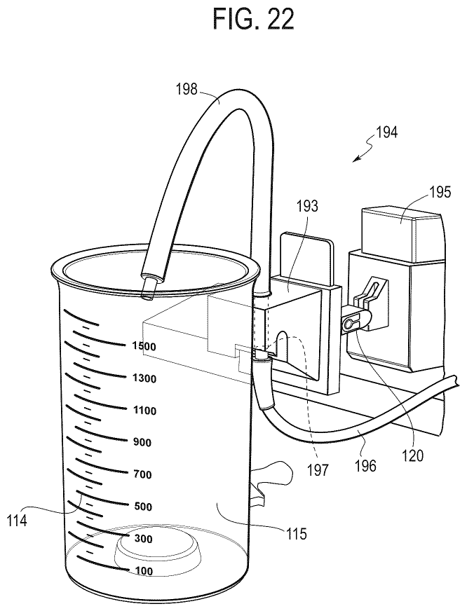

In some embodiments, for example, as shown in FIG. 22, a suspension system 194 may be used to suspend the canister 114 onto a structure (e.g., a wall) or the vacuum source 112 (e.g., as shown in FIG. 2). The sensor 120 may be operatively coupled to the suspension system 194 to detect an amount of fluid (e.g., blood) collected in the canister 114 and a rate of fluid coming into the canister 114. As shown in FIG. 22, the suspension system 194 includes a first portion 193, a second portion 195, and a sensor 120 disposed between and coupled to the first portion 193 and the second portion 195. In various examples, the sensor 120 includes a strain gauge, which measures the deflection of the suspension system 194 as fluid enters into the canister 114, however other sensor types that measure strain or weight may be suitable in other examples. The first portion 193 is configured to detachably couple to a side wall 115 of the canister 114, and the second portion 195 is configured to be attached to the structure, such that the canister 114 is suspended onto the structure. The first portion 193 may include a through hole 197, such that one or more tubes (e.g., tube 196 and tube 198, as shown in FIG. 22) may extend through the first portion 193 via the through hole 197, and allow the fluid to be pulled from the vacuum pad 104 (e.g., as shown in FIG. 2), via the tubes 196 and 198, and then flow into the canister 114.

In some embodiments, the suspension system 194 with the sensor 120 (e.g., strain gauge) may be operatively coupled with an internal clock and/or a processor or computing device, and together is configured to detect an amount of fluid collected in the canister 114 and/or a rate of fluid coming into the canister 114 (e.g., pulled from the vacuum pad 104). In some embodiments, the sensor 120 may be operatively coupled (wired or wireless) to a computing device, which may include one or more processors, and which is configured to calculate the total fluid (e.g., blood) loss and fluid loss rate (e.g., volume over time) of a patient using the vacuum pad 104 based on the strain caused by, or the measured weight of, the fluid collected in the canister 114. For example, the computing device may calculate the total fluid loss by calculating a difference in weight or strain amount from an initial starting point (e.g., a calibrated strain or weight when the canister 114 is empty) and a current weight or strain amount, which the computing device can then convert from a weight or strain amount to a fluid measurement (e.g., milliliters) using a conversion formula. The computing device may also calculate the rate of fluid loss by calculating an amount of fluid added to the canister 114 over a time period (e.g., per second, per minute, per hour). The calculated total fluid loss amount and/or the calculated rate of fluid loss may be recalculated and updated periodically, such as every second, every five seconds, every 10 seconds, every minute, etc. The computing device may then cause the calculated total fluid loss amount and/or the calculated rate of fluid loss to be displayed on a display device such as a display screen or other visual readout device, and may update the calculated value periodically as calculated. The computing device may also be configured to compare the calculated total fluid loss amount and/or the calculated rate of fluid loss to one or more threshold value, and if the calculated total fluid loss amount and/or the calculated rate of fluid loss exceeds an associated threshold value, the computing device may cause an alarm to be output, such as a visual alarm displayed on the display device or an audible alarm.

The specific configurations (e.g., the shape, size) of the suspension system 194 may be varied, as desired and/or needed, to accommodate different configurations (e.g., shape, size) of the canister 114, and the structure onto which the canister 114 is suspended, without departing from the scope of the present disclosure. The suspension system 194 may work with any canister size (500, 1000, 1500 mL, etc.) by calibrating the sensor 120 (e.g., strain gauge) when the canister 114 is suspended in the system and empty.

When coupled within a monitor/alarm system, a computer of the monitor/alarm system may be electrically connected to the sensor 120 such that the monitor/alarm system evaluates the blood collection and compare such collection to particular quantity and/or rate thresholds. If a preset threshold is met, the monitor/alarm system may provide an alert, such as by sounding a visual or audible alarm or by otherwise notifying the medical professional(s) of potential problem. The monitor/alarm system may additionally, or alternatively, include data collection and evaluation capabilities such that the procedure can be evaluated after the fact.

In addition to the depicted sensor 120, the canister 114 may include one or more sensors or other devices for detecting other characteristics of the blood or other fluid. For example, the system may be capable of detecting and monitoring a visual characteristic (e.g., color, a viscosity of the fluid, a conductivity of the fluid, temperature of the fluid, a salinity of the fluid, an acidity of the fluid, or any other characteristic where evaluation is desirable.

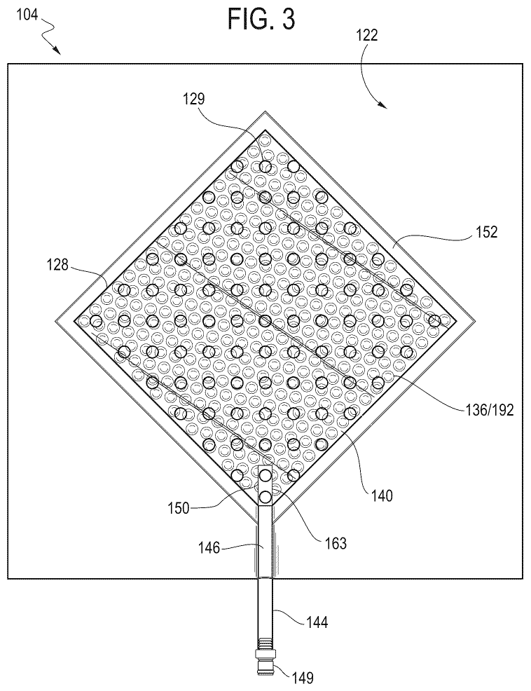

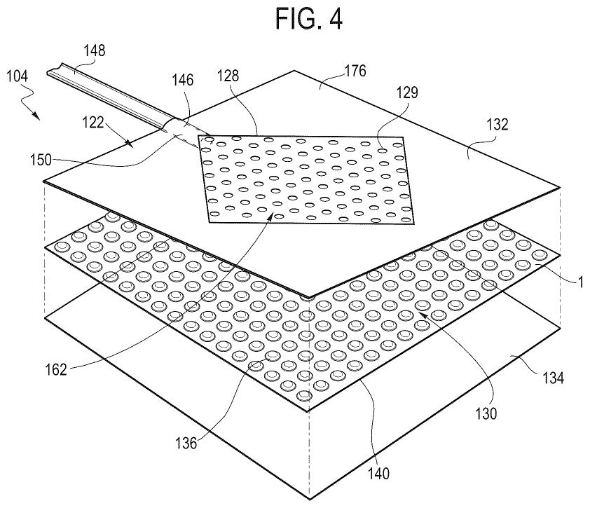

FIGS. 3-4 show the vacuum pad 104 in isolation. During a medical procedure, the upper surface 122 may face the patient body, and the lower surface 124 may face opposite the upper surface 122 (e.g., such that the lower surface 124 may contact a surgery table). An active area 128 of the vacuum pad 104 may be surrounded by a boundary portion 176 (which is discussed in more detail below), where the active area 128 is generally sized and shaped for placement beneath a target area of the patient's body for fluid collection.

The vacuum pad 104 may be outfitted with drainage capability (and/or other fluid-control capability, such as absorption) for removing or otherwise addressing fluid that would otherwise collect on the vacuum pad's upper surface 122. For instance, the upper surface 122 of the vacuum pad 104 may include one or more holes 129 that extend to a chamber or interior space 130 within the vacuum pad 104. The interior space 130 may be defined between a top layer 132 and a bottom layer 134 (see FIG. 4). When blood collects on the upper surface 122, it flows through these holes 129 and into the interior space 130. This flow may be caused or assisted via the vacuum system discussed above.

The materials forming the top layer 132 and/or the bottom layer 134 may be any suitable material(s). For example, the top layer 132 may be formed of a hydrophobic material to ensure the vacuum pad 104 does not absorb any liquid, which may be advantageous for facilitating flow of the fluid towards the center of the vacuum pad 104. The bottom layer 134 may include a texturized, non-slip material on its bottom surface, which may ensure the vacuum pad 104 maintains proper placement relative to the surgical table, for example.

To form the interior space 130 inside the vacuum pad 104, the top layer 132 and the bottom layer 134 may be separated via one or more spacers 136. For example, spacers 136 may be included between the top layer 132 and the bottom layer 134 such that the interior space 130 is formed with a plurality of fluid flowpaths or channels that generally extend around and between the spacers 136. The spacers 136 may be formed with any suitable structure. For example, in a non-limiting exemplary embodiment, the spacers 136 may be air pockets surrounded by a membrane 140 (e.g., of a polymer) or another sealed structure. To ensure the spacers 136 maintain desirable relative positioning, the spacers 136 are optionally secured to one another and/or to at least one of the top layer 132 and the bottom layer 134 of the vacuum pad 104. For example, these air pockets 142 may be coupled together via one or more securement structures to form a spacer pad, in this case in a manner resembling (or identical to) a sheet of common bubble wrap. When the spacers 136 include the membrane layer 140, it is possible for the membrane layer 140 to act as the lower-most layer of the vacuum pad 104 (meaning the bottom layer 134 shown in FIG. 4, or alternatively the top layer 132, may be absent), but this is not required.

Forming the spacers 136 with resilient air pockets may be advantageous since such spacers may provide cushioning to the patient due to the compressibility of the air within, particularly when patient's body weight will at least partially rest upon the vacuum pad 104. While the spacers 136 may compress a certain amount, the spacers 136 will ideally retain their shape enough such that the channels 138 between the spacers 136 are not sealed off. However, since a relatively large active area 128 with many spacers 136 may be included, restriction or prevention of blood flow between certain adjacent spacers 136 in certain areas of the vacuum pad 104 may occur without substantially harming the overall function of the vacuum pad 104.

Additionally, or alternatively, spacers may be included that have a different construction. For example, solid spacers formed of rubber or another compliant material may be used and may have a similar cushioning effect. In situations where compressibility is not desirable (e.g., where uninterrupted fluid passage between spacers is critical), relatively rigid spacers may be used. The spacers 136 may have any suitable size, shape, orientation, relative positioning (e.g., pattern), or the like such that blood flow, cushioning, and/or other characteristics are appropriate for a particular application. For example, the spacers 136 may be located such that they are offset relative to the holes 129 (e.g., such that they do not block the holes 129). Notably, this disclosure also covers embodiments without spacers altogether, such as an embodiment where the vacuum pad 104 includes a continuous absorbent material, where one or more tubes extending through the vacuum pad 104 render the spacers unnecessary, etc.

In the depicted embodiment, the interior space 130 between the upper top layer 132 and the bottom layer 134 may be in fluid communication with the vacuum source 112 via direct fluid communication. For example, a hollow outlet tube 144 may have an internal portion 146 that is within the interior space 130 and an external portion 148 that is outside the interior space 130. The external portion 148 of the outlet tube 144 may be connectable to the vacuum hose 110 shown in FIG. 1. Optionally, the outlet tube 144 may include a particular interface connection 149, such as threads, an O-ring, etc. where the hose 110 may be connected in a non-leaking manner.

While it is contemplated that the outlet tube 144 may simply be replaced by an extended version of the vacuum hose, a separate outlet tube 144 may be advantageous where it is desirable for the outlet tube 144 to be rigid, while the vacuum hose may be better suited as a flexible hose. For example, rigidity for the internal portion 146 of the outlet tube 144 may be important for ensuring fluid communication at the outlet of the vacuum pad 104 is not interrupted, even when under a load (e.g., the weight of the patient). By contrast, flexibility within the vacuum hose may be desirable such that the hose can be moved or otherwise manipulated throughout the room to avoid obstacles, stay clear of the surgery area, etc.

To illustrate further, the internal portion 146 of the outlet tube 144 shown in FIGS. 3-4 may include a rigid length 150 that protects the structural integrity of the outlet from the active area 128. Such a feature is particularly advantageous where the spacers 136 or other features are deformable and may otherwise have the potential for blocking/sealing off the pathway to the vacuum hose. The structure of the internal portion of the outlet tube 144 may be substantially the same as the exterior portion of the outlet tube 144, or not. For example, in some embodiments, the internal portion 146 and the external portion 148 may simply be a cylindrical-shaped tube with a uniform (and common) exterior surface. For example, a section of ⅜″ diameter plastic tubing may be used without significant modification. However, in other embodiments, the internal portion 146 and the external portion 148 may be different. For example, if the internal portion 146 of the outlet tube 144 extends beneath the holes 129, it may have a matching set of openings/holes (or another permeable structure) that allow direct flow from the upper surface 122, through the holes 129, and into the outlet tube 144. By contrast, the external portion 148 of the outlet tube 144 should remain fluid-impermeable so it does not leak.

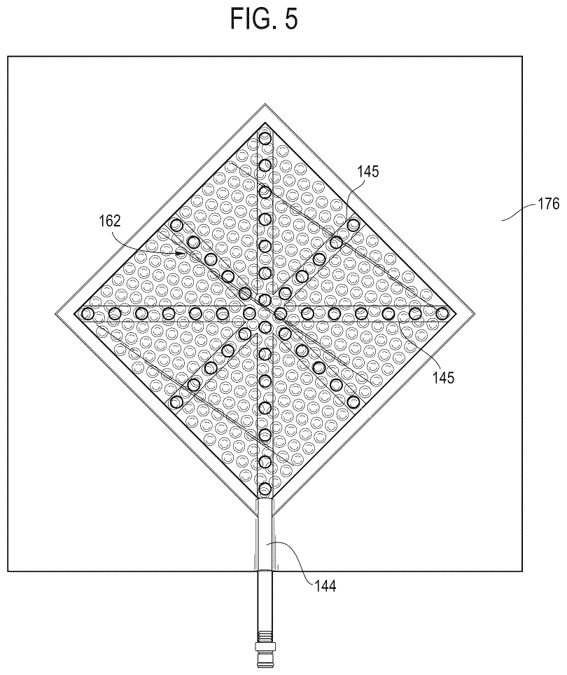

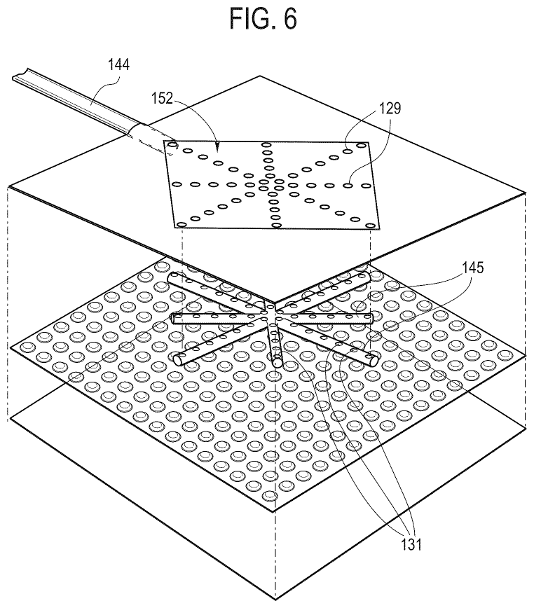

While the outlet tube 144 terminates soon after it enters the interior space 130 in FIG. 3, it may extend further in other embodiments, for example to roughly the center, or substantially across the active area 128, etc. To illustrate, referring to FIGS. 5-6, the outlet tube 144 may have one or more bifurcations where different branches 145 go different directions within the interior space 130. If the holes 129 are arranged in a particular pattern (for reasons discussed below in more detail), the outlet tube 144 may be substantially coextensive this pattern, for example, and it may include a set of holes 131 that are generally coextensive with the holes 129. Such a design may be advantageous for providing enhanced support for the interior space 130 where fluid flow rate may be critical. As mentioned above, such an embodiment may lack the need for spacers, but the spacers 136 remain included in the depicted embodiment for enhanced flow capability through the interior space 130 of the vacuum pad 104. To provide the ability for fluid to enter the outlet tube 144, the outlet tube 144 may include one or more openings, be formed of a porous material (e.g., a textile, a netting, etc.), or the like such that blood can flow into the outlet tube 144.

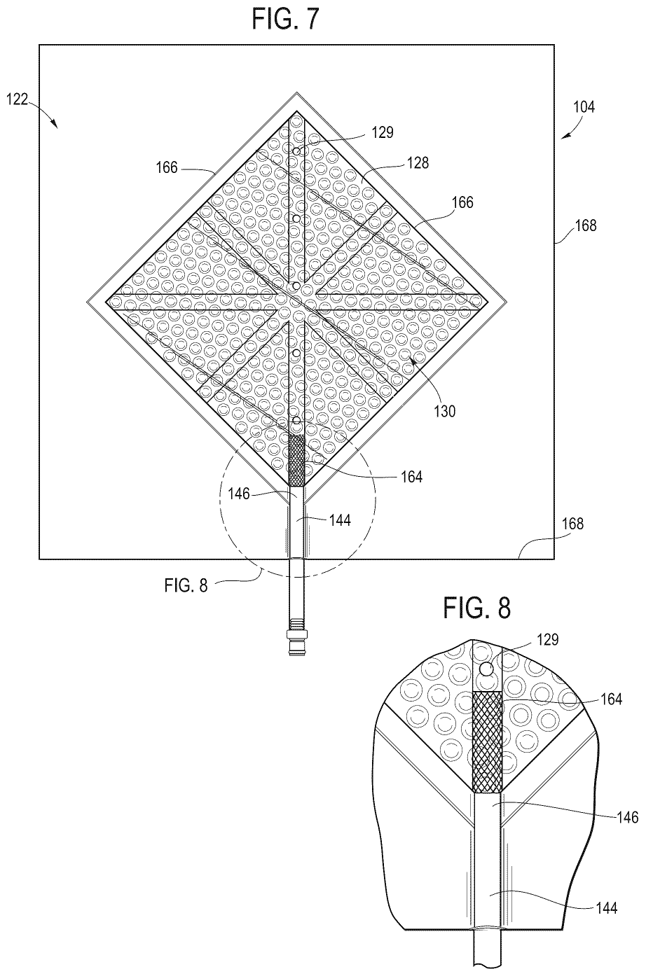

In another example, and as shown by FIGS. 7-8, one or more support structures 164 may extend from a terminal end of the outlet tube 144. As shown, the support structure 164 may include a set of protrusions, a netted/porous sleeve, or any other suitable structure that provides structural integrity to the interior space 130 at a location adjacent to the outlet tube 144. Such support structures may also/alternatively be located at other places. E.g., in a manner similar to the embodiment discussed above with reference to FIGS. 5-6, this support structure 164 may extend along a portion of, or the entirety of, a drainage pattern of the vacuum pad 104.

The active area 128 of the vacuum pad 104 may have a variety of shapes and sizes, depending on the preferred application (e.g., C-section vs. other applications), size of the patient, strength of the vacuum source, manufacturing constraints, etc. For example, the embodiments of FIGS. 1-8 each have a generally-square active area 128. While any suitable size may be used for a particular set of circumstances, the active area 128 may generally have an area of between about 3 square feet to about 0.5 square feet, such as about 1 square foot in an embodiment particularly designed for use during C-sections. The holes 129 may also be intentionally sized for particular fluid and vacuum characteristics. In the above-mentioned embodiment for C-sections, ideal hole diameter sizes range from about 0.05 centimeters to about 0.5 centimeters, such as about 0.25 centimeters. Optionally, the holes 129 may be sized to filter out certain objects (e.g., blood clots) such that the vacuum hose remains clog-free.

Notably, the performance of the device may be heavily influenced by the size of the active area 128, as well as the size, pattern, and number of holes 129. For example, during testing and using a wall vacuum source in accordance with the hospital standards noted above, a 12″ by 12″ active area having a star hole pattern (discussed below) with hole sizes mentioned above, the device provided consistent, high-performing results. In particular, the device proved capable of handling flow rates onto the upper surface 122 of up to 500 mL/min of blood without significant leakage or fluid loss. Using a 250 mL/min test flow, less than 15 mL leaked over the first 600 mL of fluid drawn into the canister.

When a square active area 128 is included, it may be advantageous for the edges 166 of the square to be angled relative to terminal edges 168 of the vacuum pad 104. For example, since it may be advantageous for the vacuum pad 104 to be limited in size, angling the square in this manner may provide enhanced “catching” ability due to a more advantageous orientation relative to the surgical site and/or target area of the patient's body.

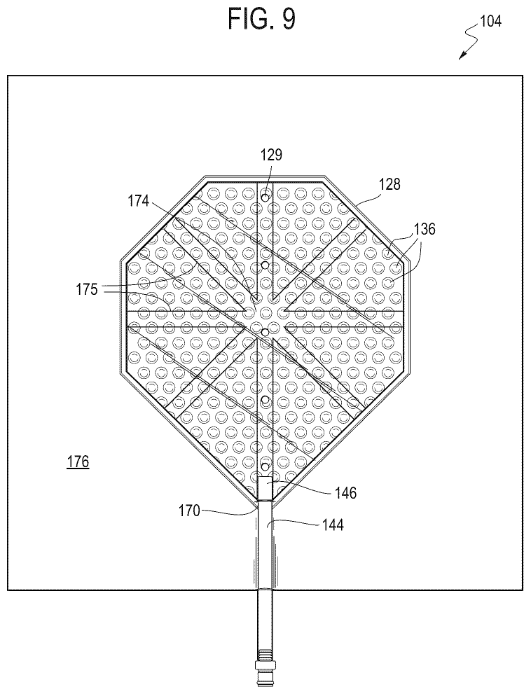

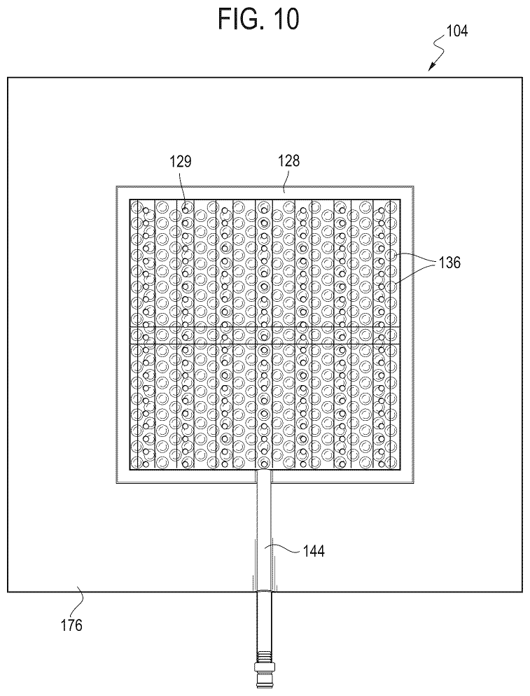

Other shapes and/or sizes of the active area 128 are additionally, or alternatively, contemplated. For example, as shown in FIG. 9, an irregular polygon (in this case an octagon) may be included with the outlet tube 144 entering at the sharpest vertex 170. FIG. 10 depicts a standard square that is not angled (in contrast to the device(s) of FIG. 1-8). Many other variations may alternatively be used. For instance, the shape of the active area 128 may be specifically designed or selected for a particular orientation and/or placement relative to the patient, and optimal shape, orientation, and/or size may vary depending on the particular procedure to be performed, size of the operating table, vacuum strength, surgeon preference, etc.

The size and/or shape of the active area 128 may also depend on the desired size of the holes 129. To illustrate, the active area 128 may be relatively larger while the hole size (or number of openings) may be relatively smaller, which may allow for a larger active area 128 without unduly sacrificing per-unit-area vacuum strength. The inverse may also be true: the size of the active area 128 may be relatively small, but the opening size (and/or number of holes 129) may be relatively large per unit area of the active area 128 such that a sufficient vacuum strength per-unit-area is possible.

The arrangement of the holes 129 may also affect the performance of the vacuum pad 104 performance, and the inventors conceived of several specific hole arrangements that improve the capabilities of the device. In particular, and as shown in each of the embodiments of FIGS. 5-9, the holes 129, the holes 129 may generally be arranged in a star pattern. Referring to FIG. 9, the star pattern may include a central area 174 along with a set of branches 175 that each extend radially outwardly from the central area 174. While any number of holes 129 may be located in each branch 175, in certain non-limiting embodiments, the holes 129 are spaced apart within the branches 175 such that about 0.3 inches to about 0.5 inches of space is located between adjacent holes 129 (and a similar spacing may be used with other patterns, for example). At least one of the branches 175 may generally align with the internal portion 146 of the outlet tube 144, which may reduce internal pressure loss as the blood moves into the outlet tube 144. While the spacers 136 are generally located throughout the active area 128, in other embodiments the spacers 136 may be concentrated beneath the locations of the holes, for example.

Other drainage patterns are also contemplated. FIG. 10 shows the holes 129 are generally placed in a grid pattern, for example, with gridlines generally parallel to the lines of the boundaries of the active area 128. FIG. 3-4, on the other hand, show a generally uniform distribution of the holes 129. May other options are also available.

Referring to FIG. 11, the vacuum pad 104 may include the defined active area 128 that is at least partially surrounded by a boundary portion 176. Optionally, the boundary portion 176 may include a construction that is substantially different than a construction of the active area 128. For example, the boundary portion 176 may generally lack the channels, spacers, and/or other features included in the active area 128 since the boundary portion 176 may not be designed to drain the fluid, although the boundary portion 176 may be configured to direct the fluid towards the active area 128 in some embodiments. For example, the boundary portion 176 may include a low-friction, water-repellant material to facilitate flow towards the center of the vacuum pad 104.

The boundary portion 176 may further include a rigidity that is greater relative to a rigidity of at least the top layer of the active area 128. For example, the boundary portion 176 may include a wood, rigid polymer/plastic, metal, or other material that causes the boundary portion 176 to retain its shape. This may be the case even when the boundary portion 176 is not placed on a surgical table. In fact, in some embodiments, the boundary portion 176 may form a portion of an upper surface 178 of the surgical table, and it is contemplated that the top of the boundary portion 176 may include cushioning and/or other features appropriate for supporting the patient's body. In certain instances, the boundary portion 176 may have a strength and rigidity sufficient such that the vacuum pad 104 may be moved while supporting the patient's body, thus having the capability to act as a transport surface for moving the patient from one place to another. When this is the case, certain indicators and/or handles may be included to indicate where lifting force should be applied. As depicted, the boundary portion 176 may be generally adjacent and co-planar to a separate support surface structure 180 of the surgical table 101, but this is not required.

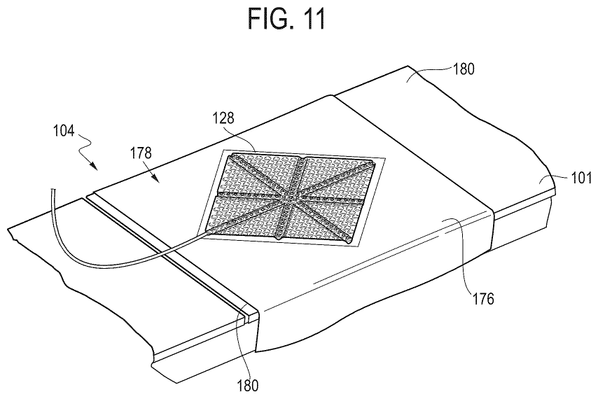

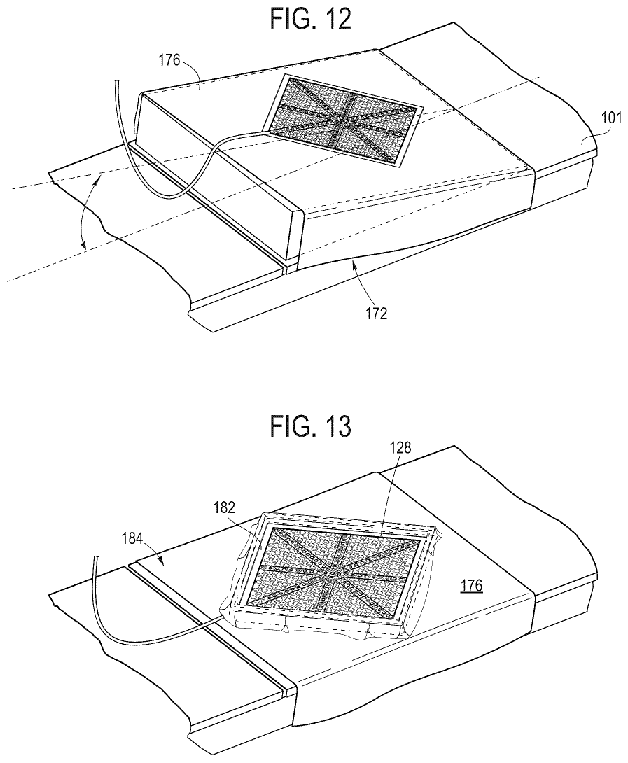

FIG. 12 shows an embodiment where the boundary portion 176 is sloped relative to the surgical table 101. The slope may be caused by an angled surface 172 located on the bottom of the boundary portion 176, which may rest on a flat surface.

As shown in FIG. 13, certain embodiments may include a raised barrier 182 that forms an interface between the active area 128 and the boundary portion 176 of the vacuum pad 104. The barrier 182 may extend from a top surface 184 of the boundary portion 176 and/or the active area 128 such that fluid collected on the active area 128 remains in place until it can be pulled away by the vacuum source. For example, in certain situations, fluid loss from the patient's body may happen at a faster rate than the active area 128 can handle in real-time, and thus the barrier 182 may form a receptacle for storing this fluid for a period of time. This may also advantageously trap blood prior to activation of the vacuum source and/or connecting the vacuum pad 104 to the hose 110.

As an alternative to the raised barrier 182, a different or additional structure may be included that accomplishes a similar function. For example, the active area 128 may be depressed relative to the top surface 184 of the boundary portion 176, for example. In other embodiments, the overall slope of the top surface 184 of the boundary area (and perhaps also the active area 128) may slope towards the center of the device. Such a slope may be caused by a particular construction causing a “default” slope (e.g., even when no patient is resting on the device), and/or the weight of the patient may create such a slope when the device is designed to conform/compress under when experiencing a load.

Referring to FIG. 14, the system 102 may optionally include a jig 190, which may be configured to direct fluid towards the active area 128 of the vacuum pad 104. For example, the jig 190 of FIG. 14 may generally fit under the sacrum and/or ischial tuberosities of a patient when used for a C-section. A central channel 192 of the jig 190 may be generally sloped towards the active area 128 such that, if and when blood flows onto the jig 190, it is directed towards the active area 128 for collection in the manner discussed above. The jig 190 may be fixed to the vacuum pad 104, but this is not required. Similarly, the jig 190 may be secured to the surgical table, or it may be freely movable.

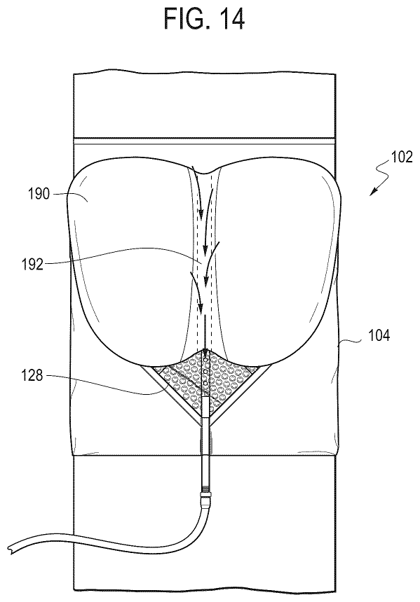

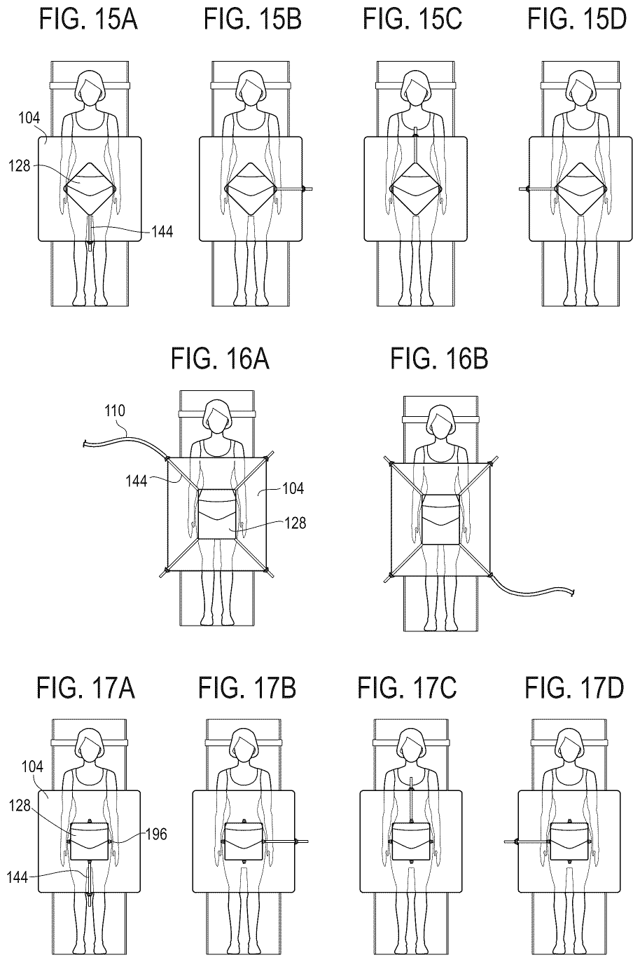

FIGS. 15A-15D show several potential locations where the outlet tube 144 may enter the active area 128 of the vacuum pad 104. For example, in each of FIGS. 14A-14D, the location of the outlet tube 144 may enter the active drainage area where two or more edges of the active area 128 meet at a corner or vertex 170. Advantageously, including the outlet tube 144 at a vertex may create a funnel effect within the interior space of the active area 128 as blood moves towards the outlet tube 144. In the embodiment of FIGS. 15A-15D, the vacuum pad 104 may be rotated such that the outlet tube 144 is in an accessible and convenient location for connection to the vacuum tube. That is, the vacuum pad 104 may work in several different orientations such that it can be placed under a patient body in a variety of ways.

In another embodiment (shown in FIGS. 15A-15B), the vacuum pad 104 may include multiple outlet tubes 144, and/or other outlet structures, such that a particular outlet structure may be chosen and connected to the vacuum hose 110 (e.g., while others go unused). In such embodiments, it may be desirable for the non-used outlet tubes to include a seal, cap, etc. such that the vacuum within the interior space is not negatively influenced. Additionally, or alternatively, multiple vacuum hoses may be coupled to multiple outlets, and/or an outlet may be used as a tap for collection of a body fluid directly from the vacuum pad 104.

FIG. 17 includes yet another embodiment where the outlet tubes 144 may be inserted into one or more ports 196. In other words, the outlet tube 144 may be connectable and disconnectable from the remainder of the vacuum pad 104 at different locations. Advantageously, when preparing for use, the vacuum pad 104 may be placed on a surgery table in a desired location without needing to account for where the outlet tube 144 or hose will be located, which may reduce the need for perfect placement (particularly helpful in emergency situations). The outlet tube 144 can then be connected in a variety of locations, even after the patient is resting on the device.

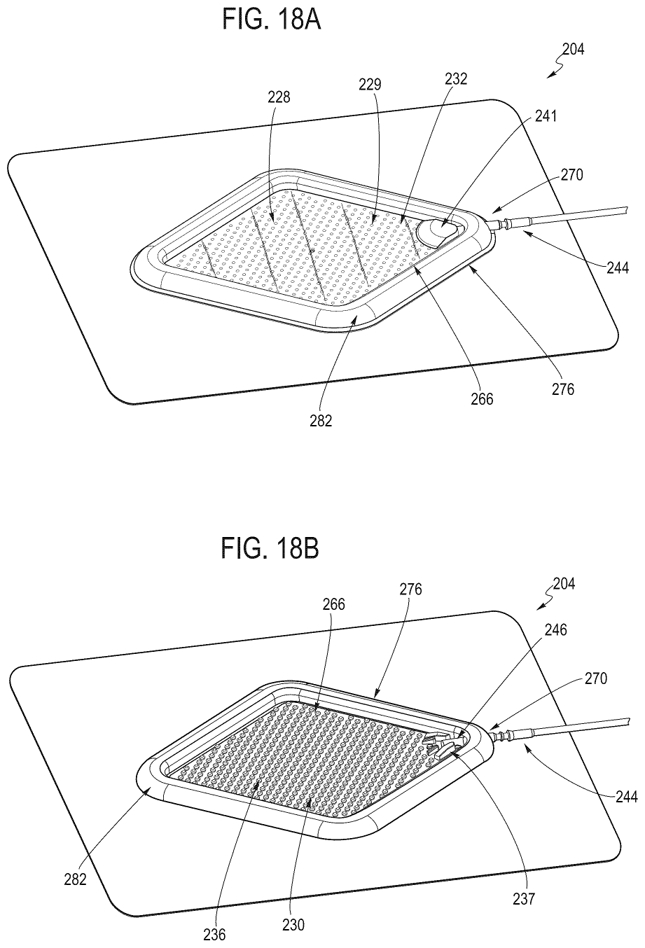

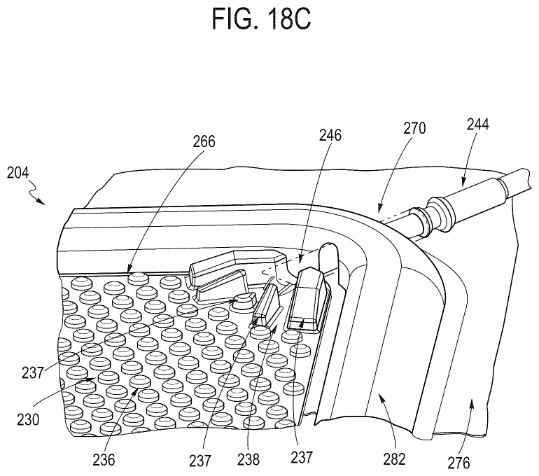

Referring to FIGS. 18A-18C, certain embodiments may include at least one divider 237 located in an interior space 230 along with at least one spacer 236. The at least one divider 237 may include a similar construction, design, and/or material as the at least one spacer 236. For example, like the at least one spacer 236, the at least one divider 237 may be formed with any suitable structure. In a non-limiting exemplary embodiment, the at least one divider 237 may be at least one air pocket surrounded by a membrane 240 (e.g., of a polymer) or another sealed structure. To ensure that the at least one divider 237 maintains desirable relative positioning, the at least one divider 237 is optionally secured to one another and/or to at least one of a top layer 232 and a bottom layer 234 of a vacuum pad 204. The at least one divider 237 may be coupled together via one or more securement structures to partially form a spacer pad with the at least one spacer, in this case in a manner resembling (or identical to) a sheet of bubble wrap. When the at least one divider 237 includes the membrane layer 240, it is possible for the membrane layer 240 to act as the lower-most layer of the vacuum pad 204 (meaning the bottom layer 234, or alternatively the top layer 232), but this is not required.

Forming the at least one divider 237 with a resilient air pocket may be advantageous since such spacers may provide cushioning to the patient due to the compressibility of the air within, particularly when patient's body weight will at least partially rest upon the vacuum pad 204. While the at least one divider 237 may compress a certain amount, the at least one divider 237 will ideally retain its shape enough such that channels 238 between the at least one divider 237 are not sealed off. However, since a relatively large active area 228 with many dividers 237 may be included, restriction or prevention of blood flow between certain adjacent dividers 237 in certain areas of the vacuum pad 204 may occur without substantially harming the overall function of the vacuum pad 204.

Additionally, or alternatively, the at least one divider 237 may be formed of a different construction and/or material. For example, the at least one divider 237 may be formed of rubber, foam, plastic, or another compliant material that provides a similar cushioning effect. In situations where compressibility is not desirable (e.g., where uninterrupted fluid passage between dividers 237 is critical), the at least one divider 237 may be relatively rigid. The at least one divider 237 may have any suitable size, shape, orientation, relative positioning (e.g., pattern), or the like such that fluid flow, cushioning, and/or other characteristics are appropriate for a particular application. For example, the at least one divider 237 may be located such that they are offset relative to the holes 229 (e.g., such that they do not block the holes 229). Notably, this disclosure also covers embodiments without the at least one divider 237 and the at least one spacer 236 altogether, such as an embodiment where the vacuum pad 204 includes a continuous absorbent material, where one or more tubes extending through the vacuum pad 204 render the spacers unnecessary, etc.

The at least one divider 237 and the at least one spacer 236 do not need to share the same characteristics. In non-limiting examples, the at least one divider 237 and the at least one spacer 236 may include different constructions, designs, materials, sizes (in thickness, length, and/or height, i.e., any dimension), shapes (including geometric features), orientations, attachments, and/or locations within the interior space 230. In the non-limiting embodiments depicted in FIGS. 18B-18C, the at least one divider 237 may have a different shape than the at least one spacer 236. Optionally, the at least one divider 237 may have an elongated shape compared to a more rounded shape of the at least one spacer 236. The at least one divider 237 may be an elongated fin as shown in FIG. 18C.

Further, the at least one divider 237 may also have a different location within the interior space 230 than the least spacer 236. Optionally, the at least one divider 237 may be adjacent to an outlet tube 244 where the at least one spacer 236 is further from the outlet tube 244. The at least one divider 237 may also be positioned among the at least one spacer 236 and/or not necessarily adjacent to the outlet tube 244 (i.e, the at least divider 237 and the at least one spacer 236 are located throughout the interior space 230 and, optionally, in particular arrangements).

In another example, the at least one divider 237 may have a height that is at least partially greater than a height of the at least one spacer 236 as shown in FIG. 18B (as well as an active area 228 and/or plurality of holes 229). As a result, the top layer 232 of the vacuum pad 204 over the at least one divider 237 may also have a height that is at least partially greater than a height of the top layer 232 over the at least one spacer 236 as shown in FIG. 18A. The at least one divider 237 may also include an at least partially sloped top surface as show in FIG. 18B. As a result, the top layer 232 of the vacuum pad 204 over the at least one divider may also include a slope as shown in FIG. 18A. In this manner, the greater height and/or sloped surface of the at least one divider 237 and the top layer 232 over the at least one divider 237 may help direct fluid flow on the top layer 232 of the vacuum pad 204 towards the plurality of holes 229 and/or the active area 228.

As shown in FIGS. 18B-18C, the at least one divider 237 may be a plurality of dividers 237. The plurality of dividers 237 may be located radially about the outlet tube 244, resulting in a fan shaped arrangement of the plurality of dividers 237. It is not required that the plurality of dividers 237 be perfectly radially about the outlet tube 244. Additionally, it is not required that the plurality of dividers 237 all and/or each need to be radially about one point along the outlet tube 244. Accordingly, the plurality of dividers 237 may be generally radial about the outlet tube 244. The plurality of dividers 237 located radially about the outlet tube 244 are generally adjacent to two edges 266 of the active area 228, to a boundary portion 276, and/or a barrier 282 and spaced apart from one another to create the channels 238. The channels 238 may extend towards the outlet tube 244 around and/or between various ones of the plurality of dividers 237. Both the plurality of dividers 237 and the channels 238 are angled towards the outlet tube 244. Each divider of the plurality of dividers 237 and the channels 238 do not need to have the same angle towards the outlet tube 244. In this manner, fluid flow is directed between and/or around the plurality of dividers 237 through the channels 238 towards the outlet tube 244.

As noted above, the at least one divider 237 and the at least one spacer 236 may not have the same size or shape. Where there is the plurality of dividers 237, the dividers 237 themselves may also have different shapes and sizes. For example, as shown in FIGS. 18B-18C, the plurality of dividers 237 may include an outer pair of dividers 237 and at least one inner divider 237. The outer pair of dividers 237 may be larger (in thickness, length, and/or height, i.e., any dimension) than the at least one divider 237. The plurality of dividers 237 may include five dividers 237, where the two outer dividers 237 are the largest, a center divider 237 is the smallest, and the two remaining dividers 237 in between the two outer dividers 237 and the center divider 237 are bigger than the center divider 237 but smaller than the outer dividers 237. Further, various ones of the at least one divider 237 may include geometric features to enhance fluid flow towards the outlet tube 244. In a non-limiting example, the center divider 237 may include a rounded surface opposite the outlet tube 244. The dividers 237 may also include a surface tapering towards the outlet tube 244 as show in FIG. 18C.

Referring to FIG. 18A, certain embodiments may include an outlet pocket 241. The outlet pocket 241 may form part of a top layer 232 of a vacuum pad 204, or be an extension of the top layer 232. The outlet pocket 241 may include a resilient space located in an interior space 230 adjacent to an outlet tube 244 between the top layer 232 and a bottom layer 234 of the vacuum pad 204. An internal portion 246 of the outlet tube 244 may terminate in an open end in the outlet pocket 241. Accordingly, the interior space 230 is continuous and in communication with the space within the outlet pocket 241 and the outlet tube 244.

The outlet pocket 241 may include a construction, design, structure, and/or materials that reduce compression near the outlet tube 244 via the resiliency of the outlet pocket 241. Optionally, the outlet pocket 241 may also provide cushioning as discussed above with a vacuum pad 204 generally. For example, the outlet pocket 241 may be formed of rubber, foam, plastic, or another compliant yet resilient material. The outlet pocket 241 may further include additional constructions, designs, materials, and/or support structures to increase resiliency. For example, as shown in FIGS. 18A-18C, at least one divider 237 may be located in the outlet pocket 241. The at least one divider 237 may be a plurality of dividers 237 in the outlet pocket 241. The plurality dividers 237 may support the outlet pocket 241 to prevent compression of the interior space 230 adjacent to the outlet tube 244. Advantageously, the plurality of dividers 237 may be angled towards the outlet tube 244 within the outlet pocket 241 to direct fluid flow towards the outlet tube 244 while the plurality of dividers 237 also support the outlet pocket 241. The outlet pocket 241 may also be supported by at least one spacer 236 in addition to, or in lieu of, the at least one divider 237.

As shown in FIG. 18A, the outlet pocket 241 may include a height relative a bottom layer 234 of a vacuum pad 204 at least partially greater than a height of the active area 228 and/or a plurality of holes 229 relative to the bottom layer 234. In this manner, the outlet pocket 241 may direct fluid flow towards the active area 228 and/or the plurality of holes 229. Additionally, the outlet pocket 241 may include at least a partially sloped top surface angled towards the active area 228 and/or the plurality of holes 229. In this manner, the outlet pocket 241 may direct fluid flow towards the active area 228 and/or the plurality of holes 229. In embodiments with the at least one divider 237, the height and/or the at least partially sloped top surface of the outlet pocket 241 may correspond to the height and/or an at least partially sloped top surface of the at least one divider 237. The top surface of the outlet pocket 241 may be impermeable to aid in directing fluid flow. In other embodiments, the top surface of the outlet pocket 241 may instead be permeable to allow fluid to flow towards the outlet tube 244 while the outlet pocket 241 also directs fluid flow towards the active area 228 and/or the plurality of holes 229.

Additionally, as illustrated in FIG. 18A, the outlet pocket 241 may include a fan shape extending from a location where the outlet tube 244 enters the interior space 230. As discussed above, the outlet tube 244 may enter the interior space 230 at various locations, including but not limited to a corner or vertex 270. The outlet pocket 241 may begin from the location and then extend and/or expand outward radially to form the fan shape. As seen in FIG. 18A, the outlet pocket 241 may be generally adjacent to and/or extend along two edges 266 of the active area 228, a boundary portion 276, and/or a barrier 282. The at least partially sloped top surface of the outlet pocket 241 may be located closer to a center of the active area 228 and/or the plurality of holes 229 than the outlet tube 244. Further, a side of the outlet pocket 241 opposite the outlet tube 244 may be rounded, consistent with the fan shape shown in FIG. 18A, or the side may be straight. Notably, the outlet pocket is not limited to a particular shape (i.e., it may be a fan shape, square or rectangular, circular, triangular, etc.). The outlet pocket 241 is also not limited to the corner or vertex 270. As noted above, the outlet tube 244 may enter the interior space 230 at various locations, such as a side of the interior space 230. The outlet pocket 241 may be also be located at various locations coinciding with the outlet tube 244 (i.e., at a side of the interior space 230).

Certain embodiments may also include an internal portion 246 of an outlet tube 244 terminating in a non-uniform manner to help minimize potential obstruction of fluid flow. In a non-limiting example, as depicted in FIG. 18C, the internal portion 246 terminates with a “V” shaped cutout on either side. Alternatively, an upper section and a lower section of the internal portion 246 extend longer than two side sections of the internal portion 246. This additional feature helps to prevent obstruction of fluid flow by providing space on the sides of the internal portion 246 for fluid to flow in the instance that a patient sitting atop the vacuum pad 204 depresses an end of the internal portion 246.

During certain medical procedures, such as a caesarian section, a surgical table may be tilted or angled relative to the ground, which, in turn, tilts a vacuum pad placed on top of the surgical table. The tilting of the surgical table may cause the fluid loss during a medical procedure to follow gravity and flow to a portion of the vacuum pad that is closest to the ground (i.e., in the direction of the tilt). Consequently, the fluid loss on top of the vacuum pad may tend to collect or pool in certain areas on top of the vacuum pad, such as along an outer portion, for example. Further, operating rooms may only provide relatively low vacuum pressure (e.g., 140 mmHg) for the vacuum pad, which may also lead to the fluid loss collecting or pooling in certain areas on top of the vacuum pad. Accordingly, there is a need for a vacuum pad having enhanced geometric features to strategically target and remove the fluid loss during a surgery.

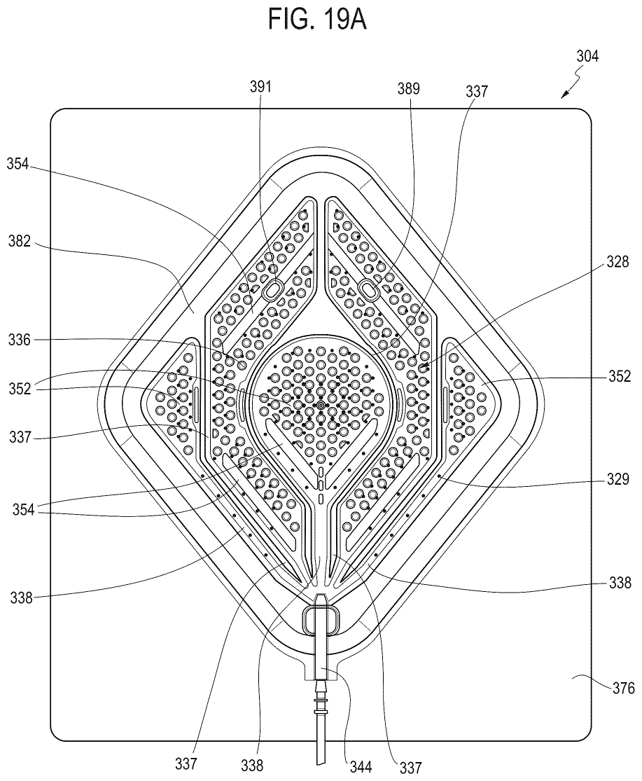

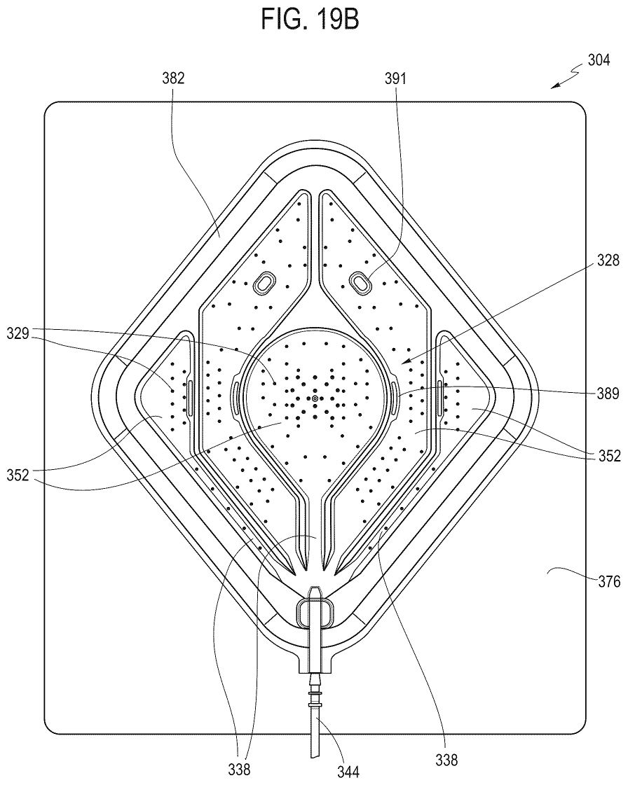

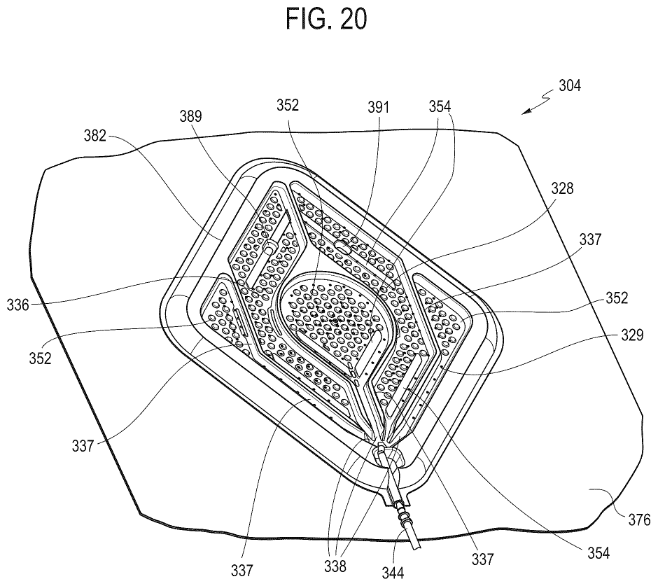

Referring to FIGS. 19A-19B, 20, and 21A-21B, another embodiment of a vacuum pad is provided. A vacuum pad 304 generally includes a first layer 334 and a top layer 332 that at least partially forms a top surface of the vacuum pad 304. The top layer 332 has a plurality of holes 329 extending through the top layer 332 into an interior space 330 that is located between at least a portion of the first layer 334 and at least a portion of the top layer 332. An outlet tube 344 is in fluid communication with the interior space 330. Like other embodiments discussed herein, the vacuum pad 304 may have at least one spacer 336 located in the interior space 330 and a raiser barrier 382 that surrounds the plurality of holes 329. Further, as illustrated in the figures, the raised barrier 382 may entirely surround the plurality of holes 329 and the at least one spacer 336 and may be elevated relative to the plurality of holes 329 and a flat boundary portion 376. The flat boundary portion 376 may include an impermeable top surface that entirely surrounds the raised barrier 382 and extends outward therefrom. As referenced for other embodiments, the vacuum pad 304 may include an active area 328 that includes the plurality of holes 329 and is at least partially surrounded by the raised barrier 182.

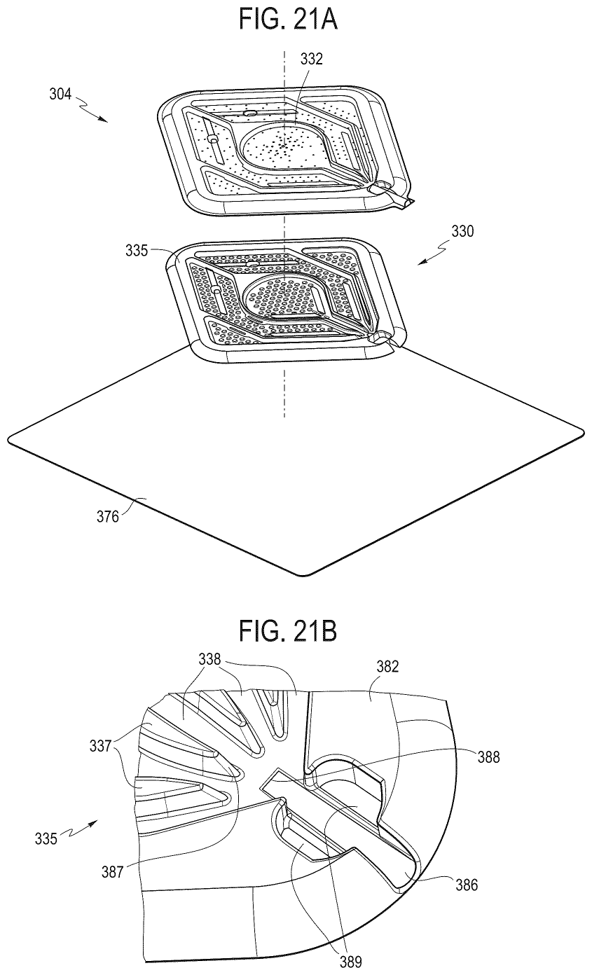

As seen in FIGS. 19A and 20, the vacuum pad 304 may include at least one divider (or one or more dividers or a plurality of dividers) 337 located in the interior space 330. Each of the dividers 337 may extend along the first layer 334 from a first end of the respective divider 337 where the first end is located adjacent to the outlet tube 344. The first ends of the dividers 337 may also be spaced radially about the outlet tube 344 in a uniform or non-uniform fashion. The one or more dividers 337 may connect to one another or to the raised barrier 382. For example, in FIG. 19A, the two centermost dividers 337 connect to one another at a location beyond a center point of the vacuum pad 304, and the outermost dividers 337 connect to the raised barrier 382. Notably, the arrangement of the one or more dividers 337 is not limited to the arrangement depicted in the exemplary figures. The one or more dividers 337 may be located elsewhere in the interior space 330 (e.g., without first ends adjacent to the outlet tube) or may not connect to one another or to the raised barrier 382. As discussed above, the one or more dividers 337 may have any suitable size, shape, orientation, relative positioning (e.g., pattern), or the like such that fluid flow, cushioning, and/or other characteristics are appropriate for a particular application. In a non-limiting example, as seen in FIG. 21B, the first end of the one or more dividers 337 may include at least one surface 387 angled or sloped towards the outlet tube 344 to direct fluid flow. Further, the one or more dividers 337 do not need to share the same shape and/or size, and the one or more dividers 337 may have a different shape than the at least one spacer 336. The one or more dividers 337 may also have a height that is about the same as a height of the raised barrier 382, or the height of the one or more dividers may be slightly more or slightly less than the height of the raised barrier 382.

Referring to FIGS. 19A and 20, the one or more dividers 337 may at least partially form at least one channel 338 that is located in the interior space 330. For example, a channel 338 may be formed by two dividers 337 or by a divider 337 and the raised barrier 382. The at least one channel 338 extends between the two dividers 337 or the divider 337 and the raised barrier 382 and towards the outlet tube 344. The at least one channel 338 also has a width between the two dividers 337 or between the divider 337 and the raised barrier 382. The width of the channel 338 adjacent to the outlet tube 344 may be equal to the height of a divider 337 that at least partially forms the channel (e.g., a 1:1 ratio of width to height), or the width of the channel 338 may be greater than or less than the height of the divider 337.

The one or more dividers 337 may also at least partially form at least one collection area 352 in the interior space 330. The at least one collection area 352 has a greater maximum width than a maximum width of the channel 338 that the collection area 352 extends from. Alternatively, a channel of the at least one channel 338 may have a first width at a section of the channel 338 adjacent to the outlet tube 344, and the channel 338 may include a section having an expanded width relative to the first width. To illustrate, FIGS. 19A and 20 depict a collection area 352 extending from a channel 338. The at least one collection area 352 has a greater maximum width than a maximum width of the channel 338. Alternatively, a channel 338 extends to a section of the channel 338 that has an expanded width relative to the first width of the channel 338 adjacent to the outlet tube 344. Further, the at least one divider 337 that at least partially forms a channel 338 may also at least partially form a collection area 352 that extends from the channel 338. For example, as seen in FIGS. 19A and 20, the two dividers 337 that form a center channel 338 also form a center collection area 352. Similarly, the divider 337 that forms an outer channel 338 with the raised barrier 382 also forms an outer collection area 352 with the raised barrier 382.

As illustrated in FIGS. 19A and 20, the vacuum pad 304 may include at least one channel 338 located in the interior space 330 that extends towards the outlet tube 344 and between one of the one or more dividers 337 and either the raised barrier 382 or another one of the one or more dividers 337. The at least one channel 338 may include a center channel 338 that is formed by two dividers of the one or more dividers 337. The at least one channel 338 may also include an outer channel 338 that is formed by another divider of the one or more dividers 337 and the raised barrier 382.

The vacuum pad 304 may further include at least one collection area 352 that extends from a channel of the at least one channel 338 and is at least partially formed by the at least one of the one or more dividers 337 that at least partially forms the channel of the at least one channel 338. The at least one collection area 352 may include a center collection area 352 that is formed by the two dividers 337 and extends from the center channel 338. The at least one collection area 352 may also include at least one outer collection area 352 that is formed by the other divider 337 and the raised barrier 382 and extends from the at least one outer channel 338. Additionally, the at least one collection area 352 may include at least one intermediary collection area 352 that is located between the center collection area 352 and the at least one outer collection area 352. The intermediary collection area 352 may be formed by one of the two dividers 337 and the other divider 337.

To describe FIGS. 19A and 20 in other words, a plurality of dividers 337 may be located in the interior space 330 with each of the plurality of dividers 337 extending along the first layer 334 from a first end of the respective divider 337 located adjacent to the outlet tube 344. A center channel 338 may be provided in the interior space 330 where the center channel 338 extends towards the outlet tube 344 and between a first divider of the plurality of dividers 337 and a second divider of the plurality of dividers 337. A center collection area 352 may be located in the interior space 330 about a center of the vacuum pad 304 and between the first divider 337 and the second divider 337. The center collection area 352 may extend from the center channel 338 in a direction away from the outlet tube 344 and have a maximum width greater than a maximum width of the center channel 338.

At least one outer channel 338 may also be located in the interior space 330 where the at least one channel 338 extends towards the outlet tube 344 and between another divider of the plurality of dividers 337 and the raised barrier 382. The other divider 337 may be located between the first divider 337 or the second divider 337 and the raised barrier 382. At least one outer collection area 352 may be located in the interior space 330 that extends from an outer channel of the at least one outer channel 338 in a direction away from the outlet tube 344. The at least one outer collection area 352 may have a maximum width greater than a maximum width of the at least one outer channel 338.

The at least one outer channel 338 may include a first outer channel 338 and a second outer channel 338, and the at least one outer collection area 352 may include a first outer collection area 352 and a second outer collection area 352. The first outer channel 338 may extend between a third divider of the plurality of dividers 337 and the raised barrier 382 to the first outer collection area 352 that is located between the third divider 337 and the raised barrier 382. The third divider 337 may be located between the first divider 337 and the raised barrier 382. The second outer channel 338 may extend between a fourth divider of the plurality of dividers 337 and the raised barrier 382 to the second outer collection area 352 that is located between the fourth divider 337 and the raised barrier 382. The fourth divider 337 may be located between the second divider 337 and the raised barrier 382.

The vacuum pad 304 may further include a first intermediary collection area 352 and a second intermediary collection area 352. The first intermediary collection area 352 may be located between the first divider 337 and the third divider 337, and the second intermediary collection area 352 may be located between the second divider 337 and the fourth divider 337. Accordingly, from left to right in FIGS. 19A and 20, the vacuum pad 304 may be provided with a first outer channel 338 that extends to a first outer collection area 352 (in between the raised barrier 382 and the third divider 337), a first intermediary collection area 352 (in between the third divider 337 and the first divider 337), a center channel 338 that extends to a center collection area 352 (in between the first divider 337 and the second divider 337), a second intermediary collection area 352 (in between the second divider 337 and the fourth divider 337), and a second outer channel 338 that extends to a second outer collection area 352 (in between the fourth divider 337 and the raised barrier 382). Additionally, by including a center collection area 352, the dividers 337 may beneficially trap fluid loss directly under where a patient is located on top of the vacuum pad 304 during a medical procedure. Outer collection areas 352, when located at corners of the raised barrier 382 (as shown in FIGS. 19A and 20), may beneficially address fluid collection or pooling towards an outer edge of the vacuum pad 304 when the vacuum pad 304 is tilted during a medical procedure. In embodiments without corners, for example, when the raised barrier 382 and/or the active area 328 is rounded, outer collection areas 352 may similarly address the fluid loss collecting or poling at an outer edge.

Notably, while FIGS. 19A-19B, 20, and 21A-21B depict this arrangement of channels 338 and collection areas 352, the vacuum pad 304 is not limited to this specific arrangement. The number of channels 338 may be greater or less than what it is illustrated in the figures, and the number of collection areas 352 may also be greater or less than what is illustrated. For example, the vacuum pad 304 may only include a center channel 338 extending to a center collection area 352 and a first outer channel 338 extending to a first outer collection area 352. The relative positioning and/or shapes of the channels 338 and the collection areas 352 may also be different. For example, a collection area 352 may be located further away from an outlet tube 344. In another example, there may be channels 338 between collection areas 352 or multiple collection areas 352 in fluid communication with one another through a channel 338.

To describe FIGS. 19A and 20 in yet another way, the vacuum pad 304 may include flow paths via the at least one channel 338 and/or at least one collection area 352 to direct fluid flow from on top of the vacuum pad 304 to the outlet tube 344. In other words, the at least one channel 338 and the at least one collection area 352 are configured to direct fluid flow to the outlet tube 344. The geometry and arrangement of the at least one channel 338 and the at least one collection area 352 may strategically target areas on the top layer 332 of the vacuum pad 304 where the fluid loss tends to collect or pool, whether due to the tilt of the vacuum pad 304 during a medical procedure or relatively weaker vacuum pressure.

As seen in the figures, the vacuum pad 304 may include three flow paths—an outer flow path, an intermediary flow path, and a center flow path. Reflected across the entire vacuum pad 304, there may be five flow paths, including two outer flow paths, two intermediary flow paths, and a center flow path. While the vacuum pad 304 is described with reference to the above arrangement of flow paths, it is not limited to this specific arrangement or number as the flow paths may positioned or sized differently to strategically target certain areas on the top layer 332 of the vacuum pad 304.

Referring to FIG. 19B, the figure is similar to FIG. 19A but with the at least one spacer 336 and a flow guide 354 (disclosed in more detail below) removed to better depict the plurality of holes 329 extending through the top layer 332 into the interior space 330. As can be seen, the plurality of holes 329 may have an arrangement where certain areas of the top layer 332 have a greater concentration, or a greater number, of the plurality of holes 329 than other areas of the top layer 332. This may allow the vacuum pad 304 to better address fluid collecting or pooling in certain areas on top of the vacuum pad 304. By way of example, a majority of the plurality of holes 329 may be located over the center collection area 352, the at least one outer collection area 352, the center channel 338, and the at least one outer channel 338. Stated differently, the top layer 332 may include a greater concentration of the plurality of holes 329 over the center channel 338, the center collection area 352, the at least one outer channel 338, and the at least one outer collection area 352 than over the at least one intermediary collection area 352. The vacuum pad 304 is not limited to the specific number of the plurality of holes 329 or the specific arrangement of the plurality of holes 329 as depicted in FIG. 19B. Rather, the figure is intended to be illustrative. The vacuum pad 304 may have a different number of the plurality of holes 329 and a different arrangement of the plurality of holes 329 than shown in FIG. 19B. The plurality of holes 329 may also have a diameter of about 0.25 centimeters, but may include a range of diameters from about 0.05 centimeters to about 0.5 centimeters. Additionally, the top layer 332 may have a degree of preforming so that the top layer 332 form fits to the features underneath (e.g., the raised barrier, one or more dividers, etc.). The top layer 332 may also have a depth that is less than a height of the one or more dividers 337 so that the top layer 332 contacts a top surface of the at least one spacer 336 and fluid may flow around and/or in between the at least one spacer 336.

Referring back to FIGS. 19A and 20, the vacuum pad 304 may also include a flow guide 354 or multiple flow guides 354 located in the interior space 330. The flow guides 354 may generally be the same height as the at least one spacer 336 and are configured to direct fluid flow within the interior space 330. The flow guides 354 may generally have a rectangular or parallelogram shape, and the shape may be different than the at least one spacer 336. As depicted in the figures, a flow guide 354 may be located in either a channel 338, a collection area 352, or both. The flow guides 354 may also be separate from the one or more dividers 337, or the flow guides 354 may be connected to a divider of the one or more dividers 337. Notably, the shape, size, number, and arrangement of the flow guides 354 are not limited to those depicted in FIGS. 19A and 20. For example, there may be more or less flow guides 354 that are arranged differently in the interior space 330 or the flow guides 354 may include multiple shapes and sizes.

Referring to FIG. 21A, certain embodiments of a vacuum pad 304 may include one or more dividers 337, at least one spacer 336, and a raised barrier 382 being formed as an insert 335, with the insert 335 being located in the interior space 330. At least one flow guide 354 may also be included in the insert 335. The insert 335 may generally be a single component that includes the features noted above as well as with additional features or with features removed. The insert 335 may be formed of or molded from foam, plastic, rubber, composite, or another material that has suitable characteristics to cushion a patient that sits on top of the vacuum pad 304 while also providing resiliency and structure for the features. The insert 335 in some embodiments may be the first layer 334 as used herein. In other embodiments, a flat boundary portion 376 may form the first layer 334, and may be, for example, positioned on top of the insert 335 and may form the first layer 334 as used elsewhere herein.

Further, as seen in FIG. 21B, the vacuum pad 304 may include a recess 386 to receive the outlet tube 344. In a non-limiting example, it is depicted as a feature of the insert 335. The recess 386 may define a lip 388 of the insert 335 such that an upper surface of the insert 335 is flush with an opening of the outlet tube 344. By providing the recess 386 with the lip 388, fluid may flow more directly to the outlet tube 344. The recess 386 may also include a ramp (i.e., an angled or sloped surface) from its starting point at or near an outer edge of the raised barrier 382 to the lip 388. For example, the insert 335 may have a thickness of about 0.1 inches at the start of the recess 386 and a thickness of about 0.25 inches at the lip 388.

Returning to FIG. 21A, a top layer 332 of the vacuum pad 304 may consist of a plastic, composite, rubber, or any other suitable material, such as urethane. As can be seen in the figure, the top layer 332 is provided on top of the insert 335 and its features. As discussed in more detail below, a non-limiting assembly of the vacuum pad 304 may include the top layer 332 attaching to the first layer (shown as flat boundary portion 376) with the insert 335 located in the interior space 330 therebetween. Alternatively, other assemblies of a vacuum pad as described herein are possible. For example, the insert 335 may include the first layer 334 of the vacuum pad 304 and the top layer 332 may be attached to the insert 335 with the features described above located on top of the first layer 334. A flat boundary portion 376 may also be attached in that instance, including instances where the flat boundary portion 376 attaches around the first layer 334 and/or underneath the first layer 334. In another example, the vacuum pad 304 may include multiple components under the top layer 332. A first component may include the one or more dividers 337 and the at least one spacer 336, and a second component may include the raised barrier 382. The first and second components may be attachable to one another. A person of ordinary skill in the art would appreciate the various assemblies of a vacuum pad as described herein.

Referring again to FIGS. 19A-19B and 20, the top layer 332 of the vacuum pad 304 may be attached, or staked, to the first layer 334 at a location 391. The top layer 332 may attached via any suitable method, including RF welding and/or with an adhesive, for example. By attaching the top layer 332 at various locations 391, the sealing of the interior space 330 may be maintained to direct fluid flow from on top of the top layer 332 through the plurality of holes 329 and the interior space 330 to the outlet tube 344. The locations 391 of attachment may be located inside and/or outside of the active area 328, including outside of the raised barrier 382.

As seen in FIGS. 19A-19B and 20, there may be locations 391 of attachment in collection areas 352, adjacent to dividers 337, and adjacent to the outlet tube 344 (discussed in greater detail below). The locations 391 of attachment may also be strategic by selecting locations where fluid is less likely to collect or pool on top of the vacuum pad 304 or where vacuum pressure is weaker in the interior space 330. For example, where a majority of the plurality of holes 329 (or a greater concentration of the plurality of holes 329 as discussed above) are located over the center collection area 352, the at least one outer collection area 352, the center channel 338, and the at least one outer channel 338, the top layer 332 may be attached to the first layer 334 at a location 391 in the active area 328 outside of the majority of the plurality of holes 329 (or where there is a lesser concentration of holes 329). In a non-limiting example, as seen FIGS. 19A-19B and 20, the location 391 may be in an intermediary collection area 352 or closer to a side of the vacuum pad 304 opposite of the outlet tube 344.

For instances where the vacuum pad 304 may include the insert 335, or another form of assembly as discussed above, the insert 335 may include at least one hole 389 at a location 391 where the top layer 332 may be attached to the first layer 334. The at least one hole 389 may extend from the upper surface of the insert 335 through to the first layer 334. Alternatively, the insert 335 may include a recess in its upper surface where the top layer 332 may be attached to the insert 335. As shown in FIG. 21B, at least one hole 389 may be located in the active area 328 as discussed above and/or adjacent to the outlet tube 344. By strategically selecting a location 391 near the outlet tube 344, the attachment of the top layer 332 further secures the outlet tube 344 against the insert 335 and/or first layer 334 so that fluid may flow more directly to the outlet tube 344.

While various embodiments have been described, it will be apparent to those of ordinary skill in the art that many more embodiments and implementations are possible. Accordingly, the embodiments described herein are examples, not the only possible embodiments and implementations.

Having described various aspects of the subject matter above, additional disclosure is provided below that may be consistent with the claims originally filed with this disclosure. In describing this additional subject matter, reference may be made to the previously described figures. Any of the following aspects may be combined, where compatible.

A first aspect includes a vacuum pad. The vacuum pad may include a first layer, a top layer that at least partially forms a top surface of the vacuum pad and has a plurality of holes extending through the top layer into an interior space that is located between at least a portion of the top layer and at least a portion of the first layer, and an outlet tube in fluid communication with the interior space. The vacuum pad may include a raised barrier that surrounds the plurality of holes, a plurality of dividers that is located in the interior space where each of the plurality of dividers extends along the first layer from a first end of the respective divider located adjacent to the outlet tube, and at least one spacer that is located in the interior space and has a different shape than the plurality of dividers. The vacuum pad may include a center channel that is located in the interior space and extends between a first divider of the plurality of dividers and a second divider of the plurality of dividers and towards the outlet tube. The vacuum pad may include a center collection area that is located in the interior space about a center of the vacuum pad and between the first divider and the second divider and extends from the center channel in a direction away from the outlet tube. The center collection area may have a greater maximum width than a maximum width of the center channel. The vacuum pad may include at least one outer channel that is located in the interior space and extends between another divider of the plurality of dividers and the raised barrier towards the outlet tube. The other divider may be located between the first divider or the second divider and the raised barrier. The vacuum pad may include at least one outer collection area that is located in the interior space between the other divider and the raised barrier and extends from an outer channel of the at least one outer channel in a direction away from the outlet tube. The at least one outer collection area may have a greater maximum width than a maximum width of the at least one outer channel.

A second aspect includes the vacuum pad from the aspect 1, where the at least one outer channel may include a first outer channel and a second outer channel, and where the at least one outer collection area may include a first outer collection area and a second outer collection area. The first outer channel may extend between a third divider of the plurality of dividers and the raised barrier to the first outer collection area that is located between the third divider and the raised barrier. The third divider may be located between the first divider and the raised barrier. The second outer channel may extend between a fourth divider of the plurality of dividers and the raised barrier to the second outer collection area that is located between the fourth divider and the raised barrier. The fourth divider may be located between the second divider and the raised barrier.

A third aspect includes the vacuum pad from the aspect 2, where the vacuum pad may include a first intermediary collection area and a second intermediary collection area. The first intermediary collection area may be located between the first divider and the third divider, and the second intermediary collection area may be located between the second divider and the fourth divider.

A fourth aspect includes the vacuum pad from any of the aspects 1-3, where a majority of the plurality of holes may be located over the center collection area, the at least one outer collection area, the center channel, and the at least one outer channel.

A fifth aspect includes the vacuum pad from any of the aspects 1-4, where the top layer may be attached to the first layer at a location.

The sixth aspect includes the vacuum pad of the aspect 5, where a majority of the plurality of holes may be located over the center collection area, the at least one outer collection area, the center channel, and the at least one outer channel, and where the raised barrier may entirely surround the plurality of holes to define an active area of the vacuum pad. The location where the top layer may be attached to the first layer may include at least one location in the active area outside of the majority of the plurality of holes.

The seventh aspect includes the vacuum pad from any of the aspects 1-6, where the first ends of the plurality of divider may be spaced radially about the outlet tube.

An eighth aspect includes a vacuum pad. The vacuum pad may include a first layer, a top layer that at least partially forms a top surface of the vacuum pad and has a plurality of holes extending through the top layer into an interior space that is located between at least a portion of the top layer and at least a portion of the first layer, and an outlet tube in fluid communication with the interior space. The vacuum pad may include a raised barrier that surrounds the plurality of holes, and one or more dividers that are located in the interior space where the one or more dividers extend along the first layer from a first end of the one or more dividers located adjacent to the outlet tube. The vacuum pad may include at least one channel that is located in the interior space and is at least partially formed by at least one of the one or more dividers. The vacuum pad may include at least one collection area that is located in the interior space and extends from a channel of the at least one channel. The at least one collection area may be at least partially formed by the at least one of the one or more dividers that at least partially forms the channel of the at least one channel. The at least one collection area may have a greater maximum width than a maximum width of the channel of the at least one channel.

A ninth aspect includes the vacuum pad of the aspect 8, where the at least one channel may include a center channel and at least one outer channel, and where the at least one collection area may include a center collection area and at least one outer collection area. The center channel may be formed by two dividers of the one or more dividers, and the at least one outer channel may be formed by another divider of the one or more dividers and the raised barrier. The center collection area may be formed by the two dividers and extend from the center channel, and the at least one outer collection area may be formed by the other divider and the raised barrier and extend from the at least one outer channel.

A tenth aspect includes the vacuum pad of the aspect 9, where the at least one collection area may include at least one intermediary collection area located between the center collection area and the at least one outer collection area. The at least one intermediary collection area may be formed by one of the two dividers and the other divider.

An eleventh aspect includes the vacuum pad of the aspect 10, where the top layer may include a greater concentration of the plurality of holes over the center channel, the center collection area, the at least one outer channel, and the at least one outer collection area than over the at least one intermediary collection area.

A twelfth aspect includes the vacuum pad from any of the aspects 8-11, where the raised barrier may entirely surround the plurality of holes and at least one spacer. The raised barrier may be elevated relative to the plurality of holes and a flat boundary portion. The flat boundary portion may include an impermeable top surface that entirely surrounds the raised barrier and extends outward therefrom.

A thirteenth aspect includes the vacuum pad from any of the aspects 8-12, where the raised barrier may include one or more corners, and where an outer collection area of the at least one outer collection area is located at a corner of the one or more corners.

A fourteenth aspect includes the vacuum pad from any of the aspects 8-13, where a channel of the at least one channel or a collection area of the at least one collection area may include a flow guide. The flow guide may be separate from the one or more dividers.

A fifteenth aspect includes a vacuum pad. The vacuum pad may include a first layer, a top layer that at least partially forms a top surface of the vacuum pad and has a plurality of holes extending through the top layer into an interior space that is located between at least a portion of the top layer and at least a portion of the first layer, and an outlet tube in fluid communication with the interior space. The vacuum pad may include a raised barrier that surrounds the plurality of holes, one or more dividers that are located in the interior space where the one or more dividers extend along the first layer from a first end of the one or more dividers located adjacent to the outlet tube, and at least one spacer that is located in the interior space and has a different shape than the plurality of dividers. The vacuum pad may include at least one channel that extends between one of the one or more dividers and either the raised barrier or another one of the one or more dividers and extends towards the outlet tube.

A sixteenth aspect includes the vacuum pad of the aspect 15, where a channel of the at least one channel has a first width at a section of the channel adjacent to the outlet tube, and where the channel includes a section having an expanded width relative to the first width.

A seventeenth aspect includes the vacuum pad from any of the aspects 15-16, where the one or more dividers, the at least one spacer, and the raised barrier may be formed as an insert. The insert may be located in the interior space.

An eighteenth aspect includes the vacuum pad of the aspect 17, where the insert may include a recess to receive the outlet tube. The recess may define a lip of the insert such that an upper surface of the insert is flush with an opening of the outlet tube.

A nineteenth aspect includes the vacuum pad from any of the aspects 17-18, where an upper surface of the insert may include at least one hole that extends through the insert to the first layer of the vacuum pad. The top layer may be attached to the first layer at a location of the at least one hole of the insert.

A twentieth aspect includes the vacuum pad from any of the aspects 15-19, where the first end of the one or more dividers may include at least one surface angled towards the outlet tube.

A twenty-first aspect includes a vacuum pad system including the vacuum pad from any of the aspects 15-19, a canister in fluid connection to the vacuum pad, wherein the canister is configured to collect fluid pulled from the vacuum pad, and a suspension system, where a side wall of the canister is detachably coupled to the suspension system, and where the suspension system is configured to detect an amount of fluid collected in the canister and/or a rate of fluid coming into the canister.

In alternative embodiments, a first aspect includes a vacuum pad. The vacuum pad may include a top layer forming a top surface of the vacuum pad; a bottom layer of the vacuum pad; an outlet tube in fluid communication with an interior space located between the top layer and the bottom layer; a plurality of holes extending through the top layer into the interior space; at least one divider located in the interior space, the at least one divider being angled towards the outlet tube; and at least one spacer located in the interior space. The at least one spacer may have a different shape than the at least one divider.

A second aspect includes the vacuum pad from the aspect 1, where the at least one divider may be adjacent to the outlet tube.

A third aspect includes the vacuum pad from any of the aspects 1-2, where the at least one divider may have a height at least partially greater than the at least one spacer, and where the top layer of the vacuum pad over the at least one divider may have a height at least partially greater than the top layer of the vacuum pad over the at least one spacer.

A fourth aspect includes the vacuum pad from any of the aspects 1-3, where the at least one divider may include an at least partially sloped top surface, and where the top layer of the vacuum pad over the at least one divider may include a slope.

A fifth aspect includes the vacuum pad from any of the aspects 1-4, where the at least one divider may include a plurality of dividers located radially about the outlet tube, and where at least one channel may extend towards the outlet tube between various ones of the plurality of dividers.

A sixth aspect includes the vacuum pad from any of the aspects 1-5, where the plurality of dividers may include an outer pair of dividers and at least one inner divider, the outer pair of dividers may be larger than the at least one inner divider.

A seventh aspect includes the vacuum pad from any of the aspects 1-6, where the at least one inner divider may include a center divider having a rounded surface opposite the outlet tube.

An eighth aspect includes the vacuum pad from any of the aspects 1-7, where the vacuum pad may include a raised barrier that entirely surrounds the plurality of holes, the plurality of dividers, and the at least one spacer. The raised barrier may be elevated relative to the plurality of holes and a flat boundary portion and configured to collect the fluid over the plurality of holes. The boundary portion may have an impermeable top surface entirely surrounding the raised barrier and extending outward therefrom.

A ninth aspect includes a vacuum pad. The vacuum pad may include an active area having an at least partially permeable top layer, a bottom layer, and an interior space located between the at least partially permeable top layer and the bottom layer; and an outlet tube configured to couple to a vacuum source such that a fluid collected on the active area is removed from the interior space towards the vacuum source. An end of the outlet tube may be located in an outlet pocket of the interior space. The outlet pocket may include a height greater than a height of the active area, the outlet pocket may be configured to direct the fluid towards the active area.

A tenth aspect includes the vacuum pad from the aspect 9, where the outlet pocket may include at least a partially sloped top surface angled towards the active area.

An eleventh aspect includes the vacuum pad from any of the aspects 9-10, where the outlet pocket may be resilient.

A twelfth aspect includes the vacuum pad from any of the aspects 9-11, where the vacuum pad may include a plurality of dividers located in the outlet pocket, the plurality of dividers may be configured to support the outlet pocket.

A thirteenth aspect includes the vacuum pad from any of the aspects 9-12, where the plurality of dividers may include a plurality of elongated fins angled toward the outlet tube.

A fourteenth aspect includes the vacuum pad from any of the aspects 9-13, where the vacuum pad may include a boundary portion that surrounds an entire perimeter of the active area. The boundary portion may include an elevated section protruding upwards relative to the active area and a flat section of the boundary portion. The flat section may entirely surround and extend outward from the elevated section.

A fifteenth aspect includes a vacuum pad. The vacuum pad may include a top layer forming a top surface of the vacuum pad; a bottom layer of the vacuum pad; an outlet tube in fluid communication with an interior space located between the top layer and the bottom layer; and a plurality of holes extending through the top layer into the interior space. An end of the outlet tube may be located in an outlet pocket of the interior space. A plurality of dividers may be located in the outlet pocket, the plurality of dividers may be angled towards the outlet tube.

A sixteenth aspect includes the vacuum pad from the aspect 15, the vacuum pad may include a plurality of spacers that have a different shape than the plurality of dividers.

A seventeenth aspect includes the vacuum pad from any of the aspects 15-16, where the plurality of dividers may be at least partially elevated relative to the plurality of holes, and where the outlet pocket may be at least partially elevated relative to the plurality of holes.

An eighteenth aspect includes the vacuum pad from any of the aspects 15-17, where the plurality of dividers may include at least one divider including an at least partially sloped surface, and where the outlet pocket may include an at least partially sloped surface angled towards the plurality of holes.

A nineteenth aspect includes the vacuum pad from any of the aspects 15-18, where at least a subset of the plurality of dividers may extend radially away from the outlet tube, and where the outlet pocket may have a fan shape extending from a location where the outlet tube enters the interior space.

A twentieth aspect includes the vacuum pad from any of the aspects 15-19, the vacuum pad may include a boundary portion that entirely surrounds the plurality of holes. The boundary portion may include a raised barrier elevated relative to the plurality of holes and a flat section of the boundary portion. The flat section may extend outward from and entirely surround the raised barrier.

In further alternative embodiments, a first aspect includes a vacuum pad. The vacuum pad may include a top layer forming a top surface of the vacuum pad; a bottom layer forming a bottom surface of the vacuum pad; and a plurality of spacers located between the top layer and the bottom layer such that an interior space is formed between the top layer and the bottom layer, the interior space having at least one channel extending around at least one spacer of the plurality of spacers; and an outlet tube in fluid communication with the interior space. The top layer may include a plurality of holes such that the vacuum pad is configured to cause a fluid collecting on the top surface of the vacuum pad to flow through the holes, into the interior space, and to the outlet tube.

A second aspect includes the vacuum pad from the aspect 1, where the vacuum pad of the first aspect may have the set of spacers of the plurality of spacers including air pockets.

A third aspect includes the vacuum pad from any of aspects 1-2, where the bottom layer of the vacuum pad of the first aspect may include a spacer pad that at least partially forms the spacers.

A fourth aspect includes the vacuum pad from any of aspects 1-3, where the plurality of openings forms a star arrangement having a central area with a plurality of branches extending radially outward therefrom.

A fifth aspect includes the vacuum pad from any of aspects 1-4, where the outlet tube includes a rigid internal portion that extends along a drainage pattern, the drainage pattern including at least a portion of the holes in the top layer.

A sixth aspect includes the vacuum pad from any of aspects 1-5, where the outlet tube enters an active area of the top layer at a vertex of the active area.

A seventh aspect includes the vacuum pad from any of aspects 1-6 where an internal portion of the outlet tube includes a permeable structure for enhancing fluid flow into the internal portion, and where an external portion of the outlet tube includes a impermeable structure for preventing leaks at a location outside the interior space of the vacuum pad.

An eighth aspect includes the vacuum pad from any of aspects 1-6, where a permeable support structure extends from the outlet tube for protecting fluid communication at a location adjacent to the outlet tube.

A ninth aspect includes the vacuum pad from any of aspects 1-8, where an active area of the vacuum pad includes the spacers, and where the vacuum pad further comprises a boundary portion at least partially surrounding the active area, the boundary portion having a construction that is different than a construction of the active area.

A tenth aspect includes vacuum pad from any of aspects 1-9, where the boundary portion includes a rigidity that is greater than a rigidity of the top layer of the active area.

An eleventh aspect includes the vacuum pad from aspect 9, where the boundary portion includes at least one sloped surface for causing a fluid to flow towards the active area when fluid collects on the boundary portion.

A twelfth aspect includes the vacuum pad from any of aspects 9-10, further including a barrier located between the boundary portion and the active area, the boundary portion protruding upwards relative to the top surface of the active area.

A thirteenth aspect includes another embodiment of a vacuum pad. The vacuum pad may include an active area having a permeable top layer and a bottom layer such that an interior space is formed between the top layer and the bottom layer; a boundary portion surrounding the active area, the boundary portion having an impermeable top surface; and an outlet configured to couple to a vacuum source such that fluid collected on the active area is removed from the interior space towards the vacuum source.

A fourteenth aspect includes the vacuum pad aspect 13, where the boundary portion includes a sloped surface for directing the fluid towards the active area.

A fifteenth aspect includes the vacuum pad from any of aspects 13-14, where the boundary portion includes a first rectangle formed by its outer perimeter, where the active area includes a second rectangle formed by its outer perimeter, and where the first rectangle is angled relative to the second rectangle.

A sixteenth aspect includes the vacuum pad from any of aspects 13-15, where the boundary portion is configured to at least partially form a top surface of a surgical table.

A seventeenth aspect includes the vacuum pad from any of aspects 13-16, further including a fixture having a sloped surface for directing the fluid towards the active area.

An eighteenth aspect includes a vacuum system. The vacuum system may include the following: a vacuum pad for collecting a fluid flowing onto a top surface of the vacuum pad, where the vacuum pad includes a permeable top layer, a bottom layer, and a plurality of spacers located between the top layer and the bottom layer; and a canister for storing a fluid collected by the vacuum pad, where the vacuum pad includes an outlet configured for securement to a first end of a vacuum hose, and where the canister includes an inlet configured for securement to a second end of the vacuum hose such that the vacuum hose directs the fluid collected by the vacuum pad to the canister.

A nineteenth aspect includes the vacuum system of aspect 18, where the canister is coupled to a sensor for detecting a fluid level within the canister.

A twentieth aspect includes the vacuum system of any of aspects 18-19, further including a vacuum source, where the vacuum source, the canister, the vacuum hose, and the vacuum pad are connected in series.

Any of the above-discussed aspects may be combined, where suitable.

We claim:

1. A vacuum pad, comprising:
a first layer;
a top layer at least partially forming a top surface of the vacuum pad, the top layer having a plurality of holes extending through the top layer into an interior space located between at least a portion of the top layer and at least a portion of the first layer;
an outlet tube in fluid communication with the interior space;
a raised barrier, the raised barrier surrounding the plurality of holes;
a plurality of dividers located in the interior space, each of the plurality of dividers extending along the first layer from a first end of the respective divider located adjacent to the outlet tube;
at least one spacer located in the interior space, the at least one spacer having a different shape than the plurality of dividers;
a center channel located in the interior space, the center channel extending between a first divider of the plurality of dividers and a second divider of the plurality of dividers and towards the outlet tube;
a center collection area located in the interior space about a center of the vacuum pad and between the first divider and the second divider, the center collection area extending from the center channel in a direction away from the outlet tube, the center collection area having a maximum width greater than a maximum width of the center channel;
at least one outer channel located in the interior space, the at least one outer channel extending between another divider of the plurality of dividers and the raised barrier and towards the outlet tube, the other divider being located between the first divider or the second divider and the raised barrier; and
at least one outer collection area located in the interior space between the other divider and the raised barrier, the at least one outer collection area extending from an outer channel of the at least one outer channel in a direction away from the outlet tube and having a maximum width greater than a maximum width of the at least one outer channel.

2. The vacuum pad of claim 1,
wherein the at least one outer channel includes a first outer channel and a second outer channel, and
wherein the at least one outer collection area includes a first outer collection area and a second outer collection area,
the first outer channel extending between a third divider of the plurality of dividers and the raised barrier to the first outer collection area located between the third divider and the raised barrier, the third divider being located between the first divider and the raised barrier, and
the second outer channel extending between a fourth divider of the plurality of dividers and the raised barrier to the second outer collection area located between the fourth divider and the raised barrier, the fourth divider being located between the second divider and the raised barrier.

3. The vacuum pad of claim 2, comprising a first intermediary collection area and a second intermediary collection area, the first intermediary collection area located between the first divider and the third divider, and the second intermediary collection area located between the second divider and the fourth divider.

4. The vacuum pad of claim 1, wherein a majority of the plurality of holes are located over the center collection area, the at least one outer collection area, the center channel, and the at least one outer channel.

5. The vacuum pad of claim 1, wherein the top layer is attached to the first layer at a location.

6. The vacuum pad of claim 5,
wherein a majority of the plurality of holes are located over the center collection area, the at least one outer collection area, the center channel, and the at least one outer channel, and
wherein the raised barrier entirely surrounds the plurality of holes defining an active area of the vacuum pad, and
wherein the location where the top layer is attached to the first layer includes at least one location in the active area outside of the majority of the plurality of holes.

7. The vacuum pad of claim 1, wherein the first ends of the plurality of dividers are spaced radially about the outlet tube.

8. A vacuum pad, comprising:
a first layer;
a top layer at least partially forming a top surface of the vacuum pad, the top layer having a plurality of holes extending through the top layer into an interior space located between at least a portion of the top layer and at least a portion of the first layer;
an outlet tube in fluid communication with the interior space;
a raised barrier, the raised barrier surrounding the plurality of holes;
one or more dividers located in the interior space, the one or more dividers extending along the first layer from a first end of the one or more dividers located adjacent to the outlet tube;
at least one channel located in the interior space, the at least one channel being at least partially formed by at least one of the one or more dividers; and
at least one collection area located in the interior space, the at least one collection area extending from a channel of the at least one channel and being at least partially formed by the at least one of the one or more dividers that at least partially forms the channel of the at least one channel,
wherein the at least one collection area has a greater maximum width than a maximum width of the channel of the at least one channel.

9. The vacuum pad of claim 8,
wherein the at least one channel includes a center channel and at least one outer channel,
the center channel being formed by two dividers of the one or more dividers, and the at least one outer channel being formed by another divider of the one or more dividers and the raised barrier, and
wherein the at least one collection area includes a center collection area and at least one outer collection area,
the center collection area being formed by the two dividers and extending from the center channel, and the at least one outer collection area being formed by the other divider and the raised barrier and extending from the at least one outer channel.

10. The vacuum pad of claim 9, wherein the at least one collection area includes at least one intermediary collection area located between the center collection area and the at least one outer collection area, the at least one intermediary collection area being formed by one of the two dividers and the other divider.

11. The vacuum pad of claim 10, wherein the top layer includes a greater concentration of the plurality of holes over the center channel, the center collection area, the at least one outer channel, and the at least one outer collection area than over the at least one intermediary collection area.

12. The vacuum pad of claim 9, wherein the raised barrier entirely surrounds the plurality of holes and at least one spacer, the raised barrier being elevated relative to the plurality of holes and a flat boundary portion, the flat boundary portion including an impermeable top surface that entirely surrounds the raised barrier and extends outward therefrom.

13. The vacuum pad of claim 12, wherein the raised barrier includes one or more corners, and wherein an outer collection area of the at least one outer collection area is located at a corner of the one or more corners.

14. The vacuum pad of claim 8, wherein a channel of the at least one channel or a collection area of the at least one collection area includes a flow guide, the flow guide being separate from the one or more dividers.

15. A vacuum pad, comprising:
a first layer;
a top layer at least partially forming a top surface of the vacuum pad, the top layer having a plurality of holes extending through the top layer into an interior space located between at least a portion of the top layer and at least a portion of the first layer;
an outlet tube in fluid communication with the interior space;
a raised barrier, the raised barrier surrounding the plurality of holes;
one or more dividers located in the interior space, the one or more dividers extending along the first layer from a first end of the one or more dividers located adjacent to the outlet tube;
at least one spacer located in the interior space, the at least one spacer having a different shape than the one or more dividers; and
at least one channel extending between one of the one or more dividers and either the raised barrier or another one of the one or more dividers and extending towards the outlet tube.

16. The vacuum pad of claim 15, wherein a channel of the at least one channel has a first width at a section of the channel adjacent to the outlet tube, and wherein the channel includes a section having an expanded width relative to the first width.

17. The vacuum pad of claim 15, wherein the one or more dividers, the at least one spacer, and the raised barrier are formed as an insert, the insert being located in the interior space.

18. The vacuum pad of claim 17, wherein the insert includes a recess to receive the outlet tube, the recess defining a lip of the insert such that an upper surface of the insert is flush with an opening of the outlet tube.

19. The vacuum pad of claim 17, wherein an upper surface of the insert includes at least one hole extending through the insert to the first layer of the vacuum pad, and wherein the top layer is attached to the first layer at a location of the at least one hole of the insert.

20. The vacuum pad of claim 15, wherein the first end of the one or more dividers includes at least one surface angled towards the outlet tube.

21. A vacuum pad system, comprising:
the vacuum pad of claim 15;
a canister in fluid connection to the vacuum pad, wherein the canister is configured to collect fluid pulled from the vacuum pad; and
a suspension system, wherein a side wall of the canister is detachably coupled to the suspension system, and wherein the suspension system is configured to detect at least one of an amount of fluid collected in the canister or a rate of fluid coming into the canister.
